(19) United States
(12) Patent Application Publication
(10) Pub. No.: US 2017/0121643 A1
(43) Pub. Date: May 4, 2017

(54) POLYPEPTIDES HAVING AMYLASE ACTIVITY AND POLYNUCLEOTIDES ENCODING SAME

(71) Applicant: NOVOZYMES A/S, Bagsvaerd (DK)

(72) Inventors: Iben Damager, Bagsvaerd (DK); Tine Hoff, Bagsvaerd (DK); Astrid Munch, Bagsvaerd (DK)

(73) Assignee: NOVOZYMES A/S, Bagsvaerd (DK)

(21) Appl. No.: 14/900,505

(22) PCT Filed: Jun. 23, 2014
(86) PCT No.: PCT/EP2014/063155

§ 371 Date: Dec. 21, 2015

(30) Foreign Application Priority Data
Jun. 21, 2013 (EP) 13173243.0

(51) Int. Cl. C11D 3/386; C12P 21/02; C12N 9/28; C11D 11/00; C11D 1/22

(52) U.S. Cl. CPC C11D 3/386; C11D 3/38681; C11D 11/0017; C11D 1/22; C12N 9/2417; C12Y 302/01001; C12P 21/02

(57) ABSTRACT

The present invention relates to isolated polypeptides having alpha-amylase activity and polynucleotides encoding the polypeptides. The invention also relates to nucleic acid constructs, vectors, and host cells comprising the polynucleotides as well as methods of producing and using the polypeptides.

### REFERENCE TO A SEQUENCE LISTING

This application contains a Sequence Listing in computer readable form, which is incorporated herein by reference.

### BACKGROUND OF THE INVENTION

Field of the Invention

The present invention relates to polypeptides having amylase activity and polynucleotides encoding the polypeptides. The invention also relates to nucleic acid constructs, vectors, and host cells comprising the polynucleotides as well as methods of producing and using the polypeptides.

Description of the Related Art

The present invention provides polypeptides having amylase activity and polynucleotides encoding the polypeptides.

Alpha-amylases have for many years been used in laundry where is it well-known that alpha-amylases have a beneficial effect in removal of starch containing stains.

WO95/26397 discloses alkaline Bacillus amylases having good wash performance measured at temperatures in the range of 30-60° C.

WO00/60060 and WO00/60058 discloses further bacterial alpha-amylases having good wash performance.

In recent years there has been a desire to reduce the temperature of the laundry in order to reduce the energy consumption. Lowering the temperature in laundry often means that the performance of the detergent composition and the enzyme is reduced and a lower wash performance is therefore obtained at low temperature. It is therefore desired to find new alpha-amylases having good wash performance at low temperature. Thus, it is an object of the present invention to provide alpha-amylase having good wash performance at low temperature (below 30° C.), such as e.g. at 15° C.

### SUMMARY OF THE INVENTION

The present invention relates to polypeptides having alpha-amylase activity, and having a 15/30 ratio of at least 0.50 when measured in Model detergent A.

The present invention also relates to polypeptides having alpha-amylase activity, selected from the group consisting of:

(a) a polypeptide having at least 85% sequence identity to the mature polypeptide of SEQ ID NO: 2;

(b) a polypeptide encoded by a polynucleotide that hybridizes under medium-high stringency conditions with (i) the mature polypeptide coding sequence of SEQ ID NO: 1, or (ii) the full-length complement thereof;

(c) a polypeptide encoded by a polynucleotide having at least 85% sequence identity to the mature polypeptide coding sequence of SEQ ID NO: 1;

(d) a variant of the mature polypeptide of SEQ ID NO: 2 comprising a substitution, deletion, and/or insertion at one or more positions;

(e) a polypeptide having at least 85% sequence identity to the mature polypeptide of SEQ ID NO: 6;

(f) a polypeptide encoded by a polynucleotide that hybridizes under medium-high stringency conditions with (i) the mature polypeptide coding sequence of SEQ ID NO: 5, or (ii) the full-length complement thereof;

(g) a polypeptide encoded by a polynucleotide having at least 85% sequence identity to the mature polypeptide coding sequence of SEQ ID NO: 5;

(h) a variant of the mature polypeptide of SEQ ID NO: 6 comprising a substitution, deletion, and/or insertion at one or more positions;

(i) a polypeptide having at least 85% sequence identity to the mature polypeptide of SEQ ID NO: 11;

(j) a polypeptide encoded by a polynucleotide that hybridizes under medium-high stringency conditions with (i) the mature polypeptide coding sequence of SEQ ID NO: 10, or (ii) the full-length complement thereof;

(k) a polypeptide encoded by a polynucleotide having at least 85% sequence identity to the mature polypeptide coding sequence of SEQ ID NO: 10;

(l) a variant of the mature polypeptide of SEQ ID NO: 11 comprising a substitution, deletion, and/or insertion at one or more positions; and

(m) a fragment of the polypeptide of (a), (b), (c), (d), (e), (f), (g), (h), (i), (j), (k) or (l) that has alpha-amylase activity.

The present invention also relates to compositions comprising said polypeptide, in particular to detergent compositions; to isolated polynucleotides encoding the polypeptides of the present invention; nucleic acid constructs; recombinant expression vectors; recombinant host cells comprising the polynucleotides; and methods of producing the polypeptides.

The present invention also relates to methods of laundering textiles, in particular at low temperature.

### DEFINITIONS

Alpha-amylase activity: The term “alpha-amylase activity” means the activity of alpha-1,4-glucan-4-glucanohydrolases, E.C. 3.2.1.1, which constitute a group of enzymes, which catalyze hydrolysis of starch and other linear and branched 1,4-glucosidic oligo- and poly-saccharides. For purposes of the present invention, alpha-amylase activity is determined according to the procedure described in the Examples section. In one aspect, the polypeptides of the present invention have at least 20%, e.g., at least 40%, at least 50%, at least 60%, at least 70%, at least 80%, at least 90%, at least 95%, or at least 100% of the alpha-amylase activity of the mature polypeptide of SEQ ID NO: 2. In another aspect, the polypeptides of the present invention have at least 20%, e.g., at least 40%, at least 50%, at least 60%, at least 70%, at least 80%, at least 90%, at least 95%, or at least 100% of the alpha-amylase activity of the mature polypeptide of SEQ ID NO: 6. In one aspect, the polypeptides of the present invention have at least 20%, e.g., at least 40%, at least 50%, at least 60%, at least 70%, at least 80%, at least 90%, at least 95%, or at least 100% of the alpha-amylase activity of the mature polypeptide of SEQ ID NO: 11.

Allelic variant: The term “allelic variant” means any of two or more alternative forms of a gene occupying the same chromosomal locus. Allelic variation arises naturally through mutation, and may result in polymorphism within populations. Gene mutations can be silent (no change in the encoded polypeptide) or may encode polypeptides having altered amino acid sequences. An allelic variant of a polypeptide is a polypeptide encoded by an allelic variant of a gene.

cDNA: The term “cDNA” means a DNA molecule that can be prepared by reverse transcription from a mature, spliced, mRNA molecule obtained from a eukaryotic or prokaryotic cell. cDNA lacks intron sequences that may be present in the corresponding genomic DNA. The initial, primary RNA transcript is a precursor to mRNA that is processed through a series of steps, including splicing, before appearing as mature spliced mRNA.

Coding sequence: The term “coding sequence” means a polynucleotide, which directly specifies the amino acid sequence of a polypeptide. The boundaries of the coding sequence are generally determined by an open reading frame, which begins with a start codon such as ATG, GTG, or TTG and ends with a stop codon such as TAA, TAG, or TGA. The coding sequence may be a genomic DNA, cDNA, synthetic DNA, or a combination thereof.

Control sequences: The term “control sequences” means nucleic acid sequences necessary for expression of a polynucleotide encoding a mature polypeptide of the present invention. Each control sequence may be native (i.e., from the same gene) or foreign (i.e., from a different gene) to the polynucleotide encoding the polypeptide or native or foreign to each other. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the polynucleotide encoding a polypeptide.

Expression: The term “expression” includes any step involved in the production of a polypeptide including, but not limited to, transcription, post-transcriptional modification, translation, post-translational modification, and secretion.

Expression vector: The term “expression vector” means a linear or circular DNA molecule that comprises a polynucleotide encoding a polypeptide and is operably linked to control sequences that provide for its expression.

Fragment: The term “fragment” means a polypeptide having one or more (e.g., several) amino acids absent from the amino and/or carboxyl terminus of a mature polypeptide or domain; wherein the fragment has alpha-amylase activity. In one aspect, a fragment contains at least 340 amino acid residues (e.g., amino acids 50 to 389 of SEQ ID NO: 2), at least 360 amino acid residues (e.g., amino acids 40 to 399 of SEQ ID NO: 2), or at least 380 amino acid residues (e.g., amino acids 30 to 409 of SEQ ID NO: 2).

Host cell: The term “host cell” means any cell type that is susceptible to transformation, transfection, transduction, or the like with a nucleic acid construct or expression vector comprising a polynucleotide of the present invention. The term “host cell” encompasses any progeny of a parent cell that is not identical to the parent cell due to mutations that occur during replication.

Isolated: The term “isolated” means a substance in a form or environment that does not occur in nature. Non-limiting examples of isolated substances include (1) any non-naturally occurring substance, (2) any substance including, but not limited to, any enzyme, variant, nucleic acid, protein, peptide or cofactor, that is at least partially removed from one or more or all of the naturally occurring constituents with which it is associated in nature; (3) any substance modified by the hand of man relative to that substance found in nature; or (4) any substance modified by increasing the amount of the substance relative to other components with which it is naturally associated (e.g., recombinant production in a host cell; multiple copies of a gene encoding the substance; and use of a stronger promoter than the promoter naturally associated with the gene encoding the substance).

Mature polypeptide: The term “mature polypeptide” means a polypeptide in its final form following translation and any post-translational modifications, such as N-terminal processing, C-terminal truncation, glycosylation, phosphorylation, etc. In one aspect, the mature polypeptide is amino acids 24 to 428 of SEQ ID NO: 2. In another aspect, the mature polypeptide of SEQ ID NO:6 is amino acids 1-409. In another aspect, the mature polypeptide of SEQ ID NO:11 is amino acids 1-409. It is known in the art that a host cell may produce a mixture of two of more different mature polypeptides (i.e., with a different C-terminal and/or N-terminal amino acid) expressed by the same polynucleotide. It is also known in the art that different host cells process polypeptides differently, and thus, one host cell expressing a polynucleotide may produce a different mature polypeptide (e.g., having a different C-terminal and/or N-terminal amino acid) as compared to another host cell expressing the same polynucleotide.

Mature polypeptide coding sequence: The term “mature polypeptide coding sequence” means a polynucleotide that encodes a mature polypeptide having alpha-amylase activity. In one aspect, the mature polypeptide coding sequence is nucleotides 70 to 1284 of SEQ ID NO: 1. Nucleotides 1 to 69 of SEQ ID NO: 1 encode a signal peptide. In another aspect, the mature polypeptide coding sequence is nucleotides 67 to 1293 of SEQ ID NO: 5. Nucleotides 1 to 66 of SEQ ID NO: 5 encode a signal peptide. In yet another aspect of the invention, the mature polypeptide coding sequence is nucleotides 67 to 1293 of SEQ ID NO: 10. Nucleotides 1 to 67 of SEQ ID NO: 10 encode a signal peptide.

Nucleic acid construct: The term “nucleic acid construct” means a nucleic acid molecule, either single- or double-stranded, which is isolated from a naturally occurring gene or is modified to contain segments of nucleic acids in a manner that would not otherwise exist in nature or which is synthetic, which comprises one or more control sequences.

Operably linked: The term “operably linked” means a configuration in which a control sequence is placed at an appropriate position relative to the coding sequence of a polynucleotide such that the control sequence directs expression of the coding sequence.

Sequence identity: The relatedness between two amino acid sequences or between two nucleotide sequences is described by the parameter “sequence identity”.

For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the -nobrief option) is used as the percent identity and is calculated as follows:

(Identical Residues×100)/(Length of Alignment−Total Number of Gaps in Alignment)

For purposes of the present invention, the sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 5.0.0 or later. The parameters used are gap open penalty of 10, gap extension penalty of 0.5, and the EDNAFULL (EMBOSS version of NCBI NUC4.4) substitution matrix. The output of Needle labeled “longest identity” (obtained using the -nobrief option) is used as the percent identity and is calculated as follows:

(Identical Deoxyribonucleotides×100)/(Length of Alignment−Total Number of Gaps in Alignment)

Very low stringency conditions: The term “very low stringency conditions” means for probes of at least 100 nucleotides in length, prehybridization and hybridization at 42° C. in 5×SSPE, 0.3% SDS, 200 micrograms/ml sheared and denatured salmon sperm DNA, and 35% formamide, following standard Southern blotting procedures for 12 to 24 hours. The carrier material is finally washed three times each for 15 minutes using 2×SSC, 0.2% SDS at 45° C.

Low stringency conditions: The term “Low stringency conditions” means for probes of at least 100 nucleotides in length, prehybridization and hybridization at 42° C. in 5×SSPE, 0.3% SDS, 200 micrograms/ml sheared and denatured salmon sperm DNA, and 35% formamide, following standard Southern blotting procedures for 12 to 24 hours. The carrier material is finally washed three times each for 15 minutes using 2×SSC, 0.2% SDS at 55° C.

Medium stringency conditions: The term “medium stringency conditions” means for probes of at least 100 nucleotides in length, prehybridization and hybridization at 42° C. in 5×SSPE, 0.3% SDS, 200 micrograms/ml sheared and denatured salmon sperm DNA, and 35% formamide, following standard Southern blotting procedures for 12 to 24 hours. The carrier material is finally washed three times each for 15 minutes using 2×SSC, 0.2% SDS at 65° C.

Medium-high stringency conditions: The term “medium-high stringency conditions” means for probes of at least 100 nucleotides in length, prehybridization and hybridization at 42° C. in 5×SSPE, 0.3% SDS, 200 micrograms/ml sheared and denatured salmon sperm DNA, and 35% formamide, following standard Southern blotting procedures for 12 to 24 hours. The carrier material is finally washed three times each for 15 minutes using 1×SSC, 0.2% SDS at 65° C.

High stringency conditions: The term “High stringency conditions” means for probes of at least 100 nucleotides in length, prehybridization and hybridization at 42° C. in 5×SSPE, 0.3% SDS, 200 micrograms/ml sheared and denatured salmon sperm DNA, and 35% formamide, following standard Southern blotting procedures for 12 to 24 hours. The carrier material is finally washed three times each for 15 minutes using 0.5×SSC, 0.2% SDS at 65° C.

Very high stringency conditions: The term “High stringency conditions” means for probes of at least 100 nucleotides in length, prehybridization and hybridization at 42° C. in 5×SSPE, 0.3% SDS, 200 micrograms/ml sheared and denatured salmon sperm DNA, and 35% formamide, following standard Southern blotting procedures for 12 to 24 hours. The carrier material is finally washed three times each for 15 minutes using 0.3×SSC, 0.2% SDS at 65° C.

Subsequence: The term “subsequence” means a polynucleotide having one or more (e.g., several) nucleotides absent from the 5′ and/or 3′ end of a mature polypeptide coding sequence; wherein the subsequence encodes a fragment having alpha-amylase activity. In one aspect, a subsequence contains at least 1020 nucleotides (e.g., nucleotides 148 to 1167 of SEQ ID NO: 1), at least 1080 nucleotides (e.g., nucleotides 118 to 1197 of SEQ ID NO: 1), or at least 1140 nucleotides (e.g., nucleotides 88 to 1227 of SEQ ID NO: 1).

Variant: The term “variant” means a polypeptide having alpha-amylase activity comprising an alteration, i.e., a substitution, insertion, and/or deletion, at one or more (e.g., several) positions. A substitution means replacement of the amino acid occupying a position with a different amino acid; a deletion means removal of the amino acid occupying a position; and an insertion means adding an amino acid adjacent to and immediately following the amino acid occupying a position.

Enzyme Detergency benefit: The term “enzyme detergency benefit” is defined herein as the advantageous effect an enzyme may add to a detergent compared to the same detergent without the enzyme. Important detergency benefits which can be provided by enzymes are stain removal with no or very little visible soils after washing and/or cleaning, prevention or reduction of redeposition of soils released in the washing process (an effect that also is termed anti-redeposition), restoring fully or partly the whiteness of textiles which originally were white but after repeated use and wash have obtained a greyish or yellowish appearance (an effect that also is termed whitening). Textile care benefits, which are not directly related to catalytic stain removal or prevention of redeposition of soils, are also important for enzyme detergency benefits. Examples of such textile care benefits are prevention or reduction of dye transfer from one fabric to another fabric or another part of the same fabric (an effect that is also termed dye transfer inhibition or anti-backstaining), removal of protruding or broken fibers from a fabric surface to decrease pilling tendencies or remove already existing pills or fuzz (an effect that also is termed anti-pilling), improvement of the fabric-softness, colour clarification of the fabric and removal of particulate soils which are trapped in the fibers of the fabric or garment. Enzymatic bleaching is a further enzyme detergency benefit where the catalytic activity generally is used to catalyze the formation of bleaching component such as hydrogen peroxide or other peroxides.

Textile care benefit: “Textile care benefits”, which are not directly related to catalytic stain removal or prevention of redeposition of soils, are also important for enzyme detergency benefits. Examples of such textile care benefits are prevention or reduction of dye transfer from one textile to another textile or another part of the same textile (an effect that is also termed dye transfer inhibition or anti-backstaining), removal of protruding or broken fibers from a textile surface to decrease pilling tendencies or remove already existing pills or fuzz (an effect that also is termed anti-pilling), improvement of the textile-softness, colour clarification of the textile and removal of particulate soils which are trapped in the fibers of the textile. Enzymatic bleaching is a further enzyme detergency benefit where the catalytic activity generally is used to catalyze the formation of bleaching component such as hydrogen peroxide or other peroxides or other bleaching species.”

Dish washing composition: The term “dish washing composition” refers to all forms of compositions for cleaning hard surfaces. The present invention is not restricted to any particular type of dish wash composition or any particular detergent.

Textile: The term “textile” means any textile material including yarns, yarn intermediates, fibers, non-woven materials, natural materials, synthetic materials, and any other textile material, fabrics made of these materials and products made from fabrics (e.g., garments and other articles). The textile or fabric may be in the form of knits, wovens, denims, non-wovens, felts, yarns, and towelling. The textile may be cellulose based such as natural cellulosics, including cotton, flax/linen, jute, ramie, sisal or coir or manmade cellulosics (e.g. originating from wood pulp) including viscose/rayon, ramie, cellulose acetate fibers (tricell), lyocell or blends thereof. The textile or fabric may also be non-cellulose based such as natural polyamides including wool, camel, cashmere, mohair, rabbit and silk or synthetic polymers such as nylon, aramid, polyester, acrylic, polypropylene and spandex/elastane, or blends thereof as well as blends of cellulose based and non-cellulose based fibers. Examples of blends are blends of cotton and/or rayon/viscose with one or more companion material such as wool, synthetic fiber (e.g. polyamide fiber, acrylic fiber, polyester fiber, polyvinyl alcohol fiber, polyvinyl chloride fiber, polyurethane fiber, polyurea fiber, aramid fiber), and/or cellulose-containing fiber (e.g. rayon/viscose, ramie, flax/linen, jute, cellulose acetate fiber, lyocell). Fabric may be conventional washable laundry, for example stained household laundry. When the term fabric or garment is used it is intended to include the broader term textiles as well.

Hard surface cleaning: The term “Hard surface cleaning” is defined herein as cleaning of hard surfaces wherein hard surfaces may include floors, tables, walls, roofs etc. as well as surfaces of hard objects such as cars (car wash) and dishes (dish wash). Dish washing includes but are not limited to cleaning of plates, cups, glasses, bowls, cutlery such as spoons, knives, forks, serving utensils, ceramics, plastics, metals, china, glass and acrylics.

Improved wash performance: The term “improved wash performance” is defined herein as an enzyme or a blend of enzymes displaying an alteration of the wash performance of an alpha-amylase relative to the wash performance of comparable prior art alpha-amylases such as the alpha-amylase of SEQ ID NO: 15 e.g. by increased stain removal. The term “wash performance” includes wash performance in laundry but also e.g. in dish wash. The improvement in the wash performance may be quantified by calculating the so-called intensity value (Int). See also the wash performance test in Examples 5-8 herein.

Wash performance: The term “wash performance” is used as an enzyme's ability to remove stains present on the object to be cleaned during e.g. wash or hard surface cleaning. The improvement in the wash performance may be quantified by calculating the so-called intensity value (Int). See also the wash performance test in Example 3 herein.

Intensity value: The wash performance is measured as the brightness expressed as the intensity of the light reflected from the sample when illuminated with white light. When the sample is stained the intensity of the reflected light is lower, than that of a clean sample. Therefore the intensity of the reflected light can be used to measure wash performance, where a higher intensity value correlates with higher wash performance.

Color measurements are made with a professional flatbed scanner (Kodak iQsmart, Kodak) used to capture an image of the washed textile.
To extract a value for the light intensity from the scanned images, 24-bit pixel values from the image are converted into values for red, green and blue (RGB). The intensity value (Int) is calculated by adding the RGB values together as vectors and then taking the length of the resulting vector:

Int=\sqrt{r²+g²+b²}

Delta intensity: The terms “Delta intensity” or “Delta intensity value” are defined herein as the result of a intensity measurement of a test material, e.g. a swatch CS-28 (Center For Testmaterials BV, P.O. Box 120, 3133 KT Vlaardingen, the Netherlands) or a hard surface. The swatch is measured with a portion of the swatch, washed under identical conditions, as background. The delta intensity is the intensity value of the test material washed with amylase subtracting the intensity value of the test material washed without amylase.

Textile: Textile sample CS-28 (rice starch on cotton) is obtained from Center For Testmaterials BV, P.O. Box 120, 3133 KT Vlaardingen, the Netherlands.

Whiteness: The term “Whiteness” is defined herein as a broad term with different meanings in different regions and for different customers. Loss of whiteness can e.g. be due to greying, yellowing, or removal of optical brighteners/hueing agents. Greying and yellowing can be due to soil redeposition, body soils, colouring from e.g. iron and copper ions or dye transfer. Whiteness might include one or several issues from the list below: colourant or dye effects; incomplete stain removal (e.g. body soils, sebum ect.); re-deposition (greying, yellowing or other discolourations of the object) (removed soils re-associates with other part of textile, soiled or unsoiled); chemical changes in textile during application; and clarification or brightening of colours.

Colour clarification: During washing and wearing loose or broken fibers can accumulate on the surface of the fabrics. One consequence can be that the colours of the fabric appear less bright or less intense because of the surface contaminations. Removal of the loose or broken fibers from the textile will partly restore the original colours and looks of the textile. By the term “colour clarification”, as used herein, is meant the partial restoration of the initial colours of textile.”

Anti-pilling: The term “anti-pilling” denotes removal of pills from the textile surface and/or prevention of formation of pills on the textile surface.”

### DETAILED DESCRIPTION OF THE INVENTION

### Polypeptides Having Alpha-Amylase Activity

In an embodiment, the present invention relates to polypeptides having a sequence identity to the mature polypeptide of SEQ ID NO: 2 of at least 85%, at least 90%, at least 91%, at least 92%, at least 93%, at least 94%, at least 95%, at least 96%, at least 97%, at least 98%, at least 99%, or 100%, which have alpha-amylase activity. In one aspect, the polypeptides differ by up to 10 amino acids, e.g., 1, 2, 3, 4, 5, 6, 7, 8, 9, or 10, from the mature polypeptide of SEQ ID NO: 2.

In one embodiment the polypeptide of the present invention preferably comprises or consists of the amino acid sequence of SEQ ID NO: 2 or an allelic variant thereof; or is a fragment thereof having alpha-amylase activity. In another aspect, the polypeptide comprises or consists of the mature polypeptide of SEQ ID NO: 2. In another aspect, the polypeptide comprises or consists of amino acids 24 to 428 of SEQ ID NO: 2.

In another embodiment, the present invention relates to polypeptides having a sequence identity to the mature polypeptide of SEQ ID NO: 6 of at least 85%, at least 90%, at least 91%, at least 92%, at least 93%, at least 94%, at least 95%, at least 96%, at least 97%, at least 98%, at least 99%, or 100%, which have alpha-amylase activity. In one aspect, the polypeptides differ by up to 10 amino acids, e.g., 1, 2, 3, 4, 5, 6, 7, 8, 9, or 10, from the mature polypeptide of SEQ ID NO: 6.

In one embodiment the polypeptide of the present invention preferably comprises or consists of the amino acid sequence of SEQ ID NO: 6 or an allelic variant thereof; or is a fragment thereof having alpha-amylase activity. In another aspect, the polypeptide comprises or consists of the mature polypeptide of SEQ ID NO: 6. In another aspect, the polypeptide comprises or consists of amino acids 1 to 409 of SEQ ID NO: 6. In another embodiment the polypeptide has at least 85%, at least 90%, at least 91%, at least 92%, at least 93%, at least 94%, at least 95%, at least 96%, at least 97%, at least 98%, at least 99%, or 100% sequence identity to the polypeptide of SEQ ID NO: 9. In another aspect, the polypeptide comprises or consists of amino acids 1 to 415 of SEQ ID NO: 9.

In another embodiment, the present invention relates to polypeptides having a sequence identity to the mature polypeptide of SEQ ID NO: 11 of at least 85%, at least 90%, at least 91%, at least 92%, at least 93%, at least 94%, at least 95%, at least 96%, at least 97%, at least 98%, at least 99%, or 100%, which have alpha-amylase activity. In one aspect, the polypeptides differ by up to 10 amino acids, e.g., 1, 2, 3, 4, 5, 6, 7, 8, 9, or 10, from the mature polypeptide of SEQ ID NO: 11.

In an embodiment, the polypeptide of the present invention preferably comprises or consists of the amino acid sequence of SEQ ID NO: 11 or an allelic variant thereof; or is a fragment thereof having alpha-amylase activity. In another aspect, the polypeptide comprises or consists of the mature polypeptide of SEQ ID NO: 1. In another aspect, the polypeptide comprises or consists of amino acids 1 to 409 of SEQ ID NO: 11. In another embodiment the polypeptide has at least 85%, at least 90%, at least 91%, at least 92%, at least 93%, at least 94%, at least 95%, at least 96%, at least 97%, at least 98%, at least 99%, or 100% sequence identity to the polypeptide of SEQ ID NO: 14. In another aspect, the polypeptide has comprises or consists of amino acids 1 to 415 of SEQ ID NO: 14.

In an embodiment, the polypeptide has been isolated. In another embodiment, the present invention relates to apolypeptide having alpha-amylase activity encoded by a polynucleotide that hybridizes under high stringency conditions, or very high stringency conditions with (i) the mature polypeptide coding sequence of SEQ ID NO: 1, or (ii) the full-length complement thereof (Sambrook et al., 1989, Molecular Cloning, A Laboratory Manual, 2d edition, Cold Spring Harbor, N.Y.).

In another embodiment, the present invention relates to an polypeptide having alpha-amylase activity encoded by a polynucleotide that hybridizes under high stringency conditions, or very high stringency conditions with (i) the mature polypeptide coding sequence of SEQ ID NO: 5, or (ii) the full-length complement thereof.

In another embodiment, the present invention relates to an polypeptide having alpha-amylase activity encoded by a polynucleotide that hybridizes under high stringency conditions, or very high stringency conditions with (i) the mature polypeptide coding sequence of SEQ ID NO: 10, or (ii) the full-length complement thereof.

In an embodiment, the polypeptide has been isolated.

The polypeptides of the invention are suitable for cleaning purposes such as for laundry and hard surface cleaning such as dish wash including manual dish was and automated dish wash. In particular the polypeptides of the invention has high wash performance at low temperature and is particular beneficial in that the wash performance is high at both low and medium temperatures. Thus in one embodiment the polypeptide of the invention has a high ratio of wash performance at 15° C. to wash performance at 30° C., e.g. calculated as the 15/30 ratio as shown in example 5, where the ratio is calculated from the delta intensities at 15° C. and 30° C. Thus in a particular embodiment the invention relates to polypeptides having alpha-amylase activity, where the 15/30 ratio is at least 0.5, such as at least 0.60, such as at least 0.75 and preferably at least 0.8 when measured in model detergent A. Thus, the wash performance at 15° C. and the wash performance at 30° C. should be determined using model detergent A and the ratio calculated based on the delta intensities at 15° C. and 30° C. The wash performance of the polypeptides of the invention in Model detergent A may preferably be determined using the AMSA assay as described below, preferably using the enzyme concentration of 0.3 mg/L wash solution.

The polynucleotides of SEQ ID NOs: 1, 5 and 10 or a subsequence thereof, as well as the polypeptides of SEQ ID NOs: 2, 6 and 11 or a fragment thereof, may be used to design nucleic acid probes to identify and clone DNA encoding polypeptides having alpha-amylase activity from strains of different genera or species according to methods well known in the art. In particular, such probes can be used for hybridization with the genomic DNA or cDNA of a cell of interest, following standard Southern blotting procedures, in order to identify and isolate the corresponding gene therein. Such probes can be considerably shorter than the entire sequence, but should be at least 15, e.g., at least 25, at least 35, or at least 70 nucleotides in length. Preferably, the nucleic acid probe is at least 100 nucleotides in length, e.g., at least 200 nucleotides, at least 300 nucleotides, at least 400 nucleotides, at least 500 nucleotides, at least 600 nucleotides, at least 700 nucleotides, at least 800 nucleotides, or at least 900 nucleotides in length. Both DNA and RNA probes can be used. The probes are typically labeled for detecting the corresponding gene (for example, with ³²P, ³H, ³⁵S, biotin, or avidin). Such probes are encompassed by the present invention.

A genomic DNA or cDNA library prepared from such other strains may be screened for DNA that hybridizes with the probes described above and encodes a polypeptide having alpha-amylase activity. Genomic or other DNA from such other strains may be separated by agarose or polyacrylamide gel electrophoresis, or other separation techniques. DNA from the libraries or the separated DNA may be transferred to and immobilized on nitrocellulose or other suitable carrier material. In order to identify a clone or DNA that hybridizes with any of SEQ ID NOs: 1, 5, or 10 or a subsequence thereof, the carrier material is used in a Southern blot.

For purposes of the present invention, hybridization indicates that the polynucleotide hybridizes to a labeled nucleic acid probe corresponding to (i) SEQ ID NOs: 1, 5 or 10; (ii) the mature polypeptide coding sequence of SEQ ID NOs: 1, 5, or 10; (iii) the full-length complement thereof; or (iv) a subsequence thereof; under high to very high stringency conditions. Molecules to which the nucleic acid probe hybridizes under these conditions can be detected using, for example, X-ray film or any other detection means known in the art.

In one aspect, the nucleic acid probe is nucleotides 70 to 1284, nucleotides 270 to 1084, nucleotides 470 to 880, or nucleotides 550 to 800 of SEQ ID NO: 1. In another aspect, the nucleic acid probe is a polynucleotide that encodes the polypeptide of SEQ ID NO: 2; the mature polypeptide thereof; or a fragment thereof. In another aspect, the nucleic acid probe is SEQ ID NO: 1.

In another aspect, the nucleic acid probe is nucleotides 67 to 1293, nucleotides 267 to 1093, nucleotides 467 to 893, or nucleotides 550 to 800 of SEQ ID NO: 5. In another aspect, the nucleic acid probe is a polynucleotide that encodes the polypeptide of SEQ ID NO: 6; the mature polypeptide thereof; or a fragment thereof. In another aspect, the nucleic acid probe is SEQ ID NO: 5.

In another aspect, the nucleic acid probe is nucleotides 67 to 1293, nucleotides 267 to 1093, nucleotides 467 to 893, or nucleotides 550 to 800 of SEQ ID NO: 10. In another aspect, the nucleic acid probe is a polynucleotide that encodes the polypeptide of SEQ ID NO: 11; the mature polypeptide thereof; or a fragment thereof. In another aspect, the nucleic acid probe is SEQ ID NO: 10.

In another embodiment, the present invention relates to a polypeptide having alpha-amylase activity encoded by a polynucleotide having a sequence identity to the mature polypeptide coding sequence of SEQ ID NO: 1 of at least 85%, at least 90%, at least 91%, at least 92%, at least 93%, at least 94%, at least 95%, at least 96%, at least 97%, at least 98%, at least 99%, or 100%. In a further embodiment, the polypeptide has been isolated.

In another embodiment, the present invention relates to a polypeptide having alpha-amylase activity encoded by a polynucleotide having a sequence identity to the mature polypeptide coding sequence of SEQ ID NO: 5 of at least 85%, at least 90%, at least 91%, at least 92%, at least 93%, at least 94%, at least 95%, at least 96%, at least 97%, at least 98%, at least 99%, or 100%. In a further embodiment, the polypeptide has been isolated.

In another embodiment, the present invention relates to a polypeptide having alpha-amylase activity encoded by a polynucleotide having a sequence identity to the mature polypeptide coding sequence of SEQ ID NO: 10 of at least 85%, at least 90%, at least 91%, at least 92%, at least 93%, at least 94%, at least 95%, at least 96%, at least 97%, at least 98%, at least 99%, or 100%. In a further embodiment, the polypeptide has been isolated.

In another embodiment, the present invention relates to variants of the mature polypeptide of SEQ ID NO: 2 comprising a substitution, deletion, and/or insertion at one or more (e.g., several) positions. In an embodiment, the number of amino acid substitutions, deletions and/or insertions introduced into the mature polypeptide of SEQ ID NO: 2 is up to 10, e.g., 1, 2, 3, 4, 5, 6, 7, 8, 9, or 10. In another embodiment, the present invention relates to variants of the mature polypeptide of SEQ ID NO: 6 comprising a substitution, deletion, and/or insertion at one or more (e.g., several) positions. In an embodiment, the number of amino acid substitutions, deletions and/or insertions introduced into the mature polypeptide of SEQ ID NO: 6 is up to 10, e.g., 1, 2, 3, 4, 5, 6, 7, 8, 9, or 10. In another embodiment, the present invention relates to variants of the mature polypeptide of SEQ ID NO: 11 comprising a substitution, deletion, and/or insertion at one or more (e.g., several) positions. In an embodiment, the number of amino acid substitutions, deletions and/or insertions introduced into the mature polypeptide of SEQ ID NO: 11 is up to 10, e.g., 1, 2, 3, 4, 5, 6, 7, 8, 9, or 10. The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding domain.

Examples of conservative substitutions are within the groups of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions that do not generally alter specific activity are known in the art and are described, for example, by H. Neurath and R. L. Hill, 1979, In, The Proteins, Academic Press, New York. Common substitutions are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly.

Alternatively, the amino acid changes are of such a nature that the physico-chemical properties of the polypeptides are altered. For example, amino acid changes may improve the thermal stability of the polypeptide, alter the substrate specificity, change the pH optimum, and the like.

Essential amino acids in a polypeptide can be identified according to procedures known in the art, such as site-directed mutagenesis or alanine-scanning mutagenesis (Cunningham and Wells, 1989, Science 244: 1081-1085). In the latter technique, single alanine mutations are introduced at every residue in the molecule, and the resultant mutant molecules are tested for alpha-amylase activity to identify amino acid residues that are critical to the activity of the molecule. See also, Hilton et al., 1996, J. Biol. Chem. 271: 4699-4708. The active site of the enzyme or other biological interaction can also be determined by physical analysis of structure, as determined by such techniques as nuclear magnetic resonance, crystallography, electron diffraction, or photoaffinity labeling, in conjunction with mutation of putative contact site amino acids. See, for example, de Vos et al., 1992, Science 255: 306-312; Smith et al., 1992, J. Mol. Biol. 224: 899-904; Wlodaver et al., 1992, FEBS Lett. 309: 59-64. The identity of essential amino acids can also be inferred from an alignment with a related polypeptide. The catalytic residues that are essential for activity have been identified by alignment with known alpha-amylases as aspartic acid in position 189 (D189), glutamic acid in position 214 (E214) and aspartic acid in position (D283).

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241: 53-57; Bowie and Sauer, 1989, Proc. Natl. Acad. Sci. USA 86: 2152-2156; WO 95/17413; or WO 95/22625. Other methods that can be used include error-prone PCR, phage display (e.g., Lowman et al., 1991, Biochemistry 30: 10832-10837; U.S. Pat. No. 5,223,409; WO 92/06204), and region-directed mutagenesis (Derbyshire et al., 1986, Gene 46: 145; Ner et al., 1988, DNA 7: 127).

Mutagenesis/shuffling methods can be combined with high-throughput, automated screening methods to detect activity of cloned, mutagenized polypeptides expressed by host cells (Ness et al., 1999, Nature Biotechnology 17: 893-896). Mutagenized DNA molecules that encode active polypeptides can be recovered from the host cells and rapidly sequenced using standard methods in the art. These methods allow the rapid determination of the importance of individual amino acid residues in a polypeptide.

The polypeptide may be a hybrid polypeptide in which a region of one polypeptide is fused at the N-terminus or the C-terminus of a region of another polypeptide.

The polypeptide may be a fusion polypeptide or cleavable fusion polypeptide in which another polypeptide is fused at the N-terminus or the C-terminus of the polypeptide of the present invention. A fusion polypeptide is produced by fusing a polynucleotide encoding another polypeptide to a polynucleotide of the present invention. Techniques for producing fusion polypeptides are known in the art, and include ligating the coding sequences encoding the polypeptides so that they are in frame and that expression of the fusion polypeptide is under control of the same promoter(s) and terminator. Fusion polypeptides may also be constructed using intein technology in which fusion polypeptides are created post-translationally (Cooper et al., 1993, EMBO J. 12: 2575-2583; Dawson et al., 1994, Science 266: 776-779).

A fusion polypeptide can further comprise a cleavage site between the two polypeptides. Upon secretion of the fusion protein, the site is cleaved releasing the two polypeptides. Examples of cleavage sites include, but are not limited to, the sites disclosed in Martin et al., 2003, J. Ind. Microbiol. Biotechnol. 3: 568-576; Svetina et al., 2000, J. Biotechnol. 76: 245-251; Rasmussen-Wilson et al., 1997, Appl. Environ. Microbiol. 63: 3488-3493; Ward et al., 1995, Biotechnology 13: 498-503; and Contreras et al., 1991, Biotechnology 9: 378-381; Eaton et al., 1986, Biochemistry 25: 505-512; Collins-Racie et al., 1995, Biotechnology 13: 982-987; Carter et al., 1989, Proteins: Structure, Function, and Genetics 6: 240-248; and Stevens, 2003, Drug Discovery World 4: 35-48.

### Sources of Polypeptides Having Alpha-Amylase Activity

A polypeptide having alpha-amylase activity of the present invention may be obtained from microorganisms of any genus. For purposes of the present invention, the term “obtained from” as used herein in connection with a given source shall mean that the polypeptide encoded by a polynucleotide is produced by the source or by a strain in which the polynucleotide from the source has been inserted. In one aspect, the polypeptide obtained from a given source is secreted extracellularly.

The polypeptide may be a bacterial polypeptide. For example, the polypeptide may be a Gram-positive bacterial polypeptide such as a Bacillus, Clostridium, Enterococcus, Geobacillus, Lactobacillus, Lactococcus, Oceanobacillus, Staphylococcus, Streptococcus, Streptomyces, Ahrensia or Tenacibaculum polypeptide having alpha-amylase activity, or a Gram-negative bacterial polypeptide such as a Campylobacter, E. coli, Flavobacterium, Fusobacterium, Helicobacter, Ilyobacter, Neisseria, Pseudomonas, Salmonella, or Ureaplasma polypeptide.

In one aspect, the polypeptide is a Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, or Bacillus thuringiensis polypeptide.

In another aspect, the polypeptide is a Streptococcus equisimilis, Streptococcus pyogenes, Streptococcus uberis, or Streptococcus equi subsp. Zooepidemicus polypeptide.

In another aspect, the polypeptide is a Streptomyces achromogenes, Streptomyces avermitilis, Streptomyces coelicolor, Streptomyces griseus, or Streptomyces lividans polypeptide.

In another aspect, the polypeptide is a Tenacibaculum geojense polypeptide. In yet another aspect, the polypeptide is a Tenacibaculum sp-62066 polypeptide. In yet another aspect, the polypeptide is an Ahrensia sp-62069 polypeptide.

It will be understood that for the aforementioned species, the invention encompasses both the perfect and imperfect states, and other taxonomic equivalents, e.g., anamorphs, regardless of the species name by which they are known. Those skilled in the art will readily recognize the identity of appropriate equivalents.

Strains of these species are readily accessible to the public in a number of culture collections, such as the American Type Culture Collection (ATCC), Deutsche Sammlung von Mikroorganismen and Zellkulturen GmbH (DSMZ), Centraalbureau Voor Schimmelcultures (CBS), and Agricultural Research Service Patent Culture Collection, Northern Regional Research Center (NRRL).

The polypeptide may be identified and obtained from other sources including microorganisms isolated from nature (e.g., soil, composts, water, etc.) or DNA samples obtained directly from natural materials (e.g., soil, composts, water, etc.) using the above-mentioned probes. Techniques for isolating microorganisms and DNA directly from natural habitats are well known in the art. A polynucleotide encoding the polypeptide may then be obtained by similarly screening a genomic DNA or cDNA library of another microorganism or mixed DNA sample. Once a polynucleotide encoding a polypeptide has been detected with the probe(s), the polynucleotide can be isolated or cloned by utilizing techniques that are known to those of ordinary skill in the art (see, e.g., Sambrook et al., 1989, supra).

### Polynucleotides

The present invention also relates to isolated polynucleotides encoding a polypeptide of the present invention, as described herein.

The techniques used to isolate or clone a polynucleotide are known in the art and include isolation from genomic DNA or cDNA, or a combination thereof. The cloning of the polynucleotides from genomic DNA can be effected, e.g., by use of the well known polymerase chain reaction (PCR) or antibody screening of expression libraries to detect cloned DNA fragments with shared structural features. See, e.g., Innis et al., 1990, PCR: A Guide to Methods and Application, Academic Press, New York. Other nucleic acid amplification procedures such as ligase chain reaction (LCR), ligation activated transcription (LAT) and polynucleotide-based amplification (NASBA) may be used. The polynucleotides may be cloned from a strain of Tenacibaculum sp. such as a strain of Tenacibaculum geojense, or a Tenacibaculum sp-62066 or a related organism such as an Ahrensia polypeptide, such as Ahrensia sp-62069 and thus, for example, may be an allelic or species variant of the polypeptide encoding region of the polynucleotide.

Modification of a polynucleotide encoding a polypeptide of the present invention may be necessary for synthesizing polypeptides substantially similar to the polypeptide. The term “substantially similar” to the polypeptide refers to non-naturally occurring forms of the polypeptide. These polypeptides may differ in some engineered way from the polypeptide isolated from its native source, e.g., variants that differ in specific activity, thermostability, pH optimum, or the like. The variants may be constructed on the basis of the polynucleotide presented as the mature polypeptide coding sequence of SEQ ID NOs: 1, 5 or 10, e.g., a subsequence thereof, and/or by introduction of nucleotide substitutions that do not result in a change in the amino acid sequence of the polypeptide, but which correspond to the codon usage of the host organism intended for production of the enzyme, or by introduction of nucleotide substitutions that may give rise to a different amino acid sequence. For a general description of nucleotide substitution, see, e.g., Ford et al., 1991, Protein Expression and Purification 2: 95-107.

### Nucleic Acid Constructs

The present invention also relates to nucleic acid constructs comprising a polynucleotide of the present invention operably linked to one or more control sequences that direct the expression of the coding sequence in a suitable host cell under conditions compatible with the control sequences.

The polynucleotide may be manipulated in a variety of ways to provide for expression of the polypeptide. Manipulation of the polynucleotide prior to its insertion into a vector may be desirable or necessary depending on the expression vector. The techniques for modifying polynucleotides utilizing recombinant DNA methods are well known in the art.

The control sequence may be a promoter, a polynucleotide that is recognized by a host cell for expression of a polynucleotide encoding a polypeptide of the present invention. The promoter contains transcriptional control sequences that mediate the expression of the polypeptide. The promoter may be any polynucleotide that shows transcriptional activity in the host cell including mutant, truncated, and hybrid promoters, and may be obtained from genes encoding extracellular or intracellular polypeptides either homologous or heterologous to the host cell.

Examples of suitable promoters for directing transcription of the nucleic acid constructs of the present invention in a bacterial host cell are the promoters obtained from the Bacillus amyloliquefaciens alpha-amylase gene (amyQ), Bacillus licheniformis alpha-amylase gene (amyL), Bacillus licheniformis penicillinase gene (penP), Bacillus stearothermophilus maltogenic amylase gene (amyM), Bacillus subtilis levansucrase gene (sacB), Bacillus subtilis xyIA and xyIB genes, Bacillus thuringiensis cryIIIA gene (Agaisse and Lereclus, 1994, Molecular Microbiology 13: 97-107), E. coli lac operon, E. coli trc promoter (Egon et al., 1988, Gene 69: 301-315), Streptomyces coelicolor agarase gene (dagA), and prokaryotic beta-lactamase gene (Villa-Kamaroff et al., 1978, Proc. Natl. Acad. Sci. USA 75: 3727-3731), as well as the tac promoter (DeBoer et al., 1983, Proc. Natl. Acad. Sci. USA 80: 21-25). Further promoters are described in “Useful proteins from recombinant bacteria” in Gilbert et al., 1980, Scientific American 242: 74-94; and in Sambrook et al., 1989, supra. Examples of tandem promoters are disclosed in WO 99/43835.

Examples of suitable promoters for directing transcription of the nucleic acid constructs of the present invention in a filamentous fungal host cell are promoters obtained from the genes for Aspergillus nidulans acetamidase, Aspergillus niger neutral alpha-amylase, Aspergillus niger acid stable alpha-amylase, Aspergillus niger or Aspergillus awamori glucoamylase (glaA), Aspergillus oryzae TAKA amylase, Aspergillus oryzae alkaline protease, Aspergillus oryzae triose phosphate isomerase, Fusarium oxysporum trypsin-like protease (WO 96/00787), Fusarium venenatum amyloglucosidase (WO 00/56900), Fusarium venenatum Dania (WO 00/56900), Fusarium venenatum Quinn (WO 00/56900), Rhizomucor miehei lipase, Rhizomucor miehei aspartic proteinase, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase V, Trichoderma reesei xylanase I, Trichoderma reesei xylanase II, Trichoderma reesei xylanase III, Trichoderma reesei beta-xylosidase, and Trichoderma reesei translation elongation factor, as well as the NA2-tpi promoter (a modified promoter from an Aspergillus neutral alpha-amylase gene in which the untranslated leader has been replaced by an untranslated leader from an Aspergillus triose phosphate isomerase gene; non-limiting examples include modified promoters from an Aspergillus niger neutral alpha-amylase gene in which the untranslated leader has been replaced by an untranslated leader from an Aspergillus nidulans or Aspergillus oryzae triose phosphate isomerase gene); and mutant, truncated, and hybrid promoters thereof. Other promoters are described in U.S. Pat. No. 6,011,147.

In a yeast host, useful promoters are obtained from the genes for Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH1, ADH2/GAP), Saccharomyces cerevisiae triose phosphate isomerase (TPI), Saccharomyces cerevisiae metallothionein (CUP1), and Saccharomyces cerevisiae 3-phosphoglycerate kinase. Other useful promoters for yeast host cells are described by Romanos et al., 1992, Yeast 8: 423-488.

The control sequence may also be a transcription terminator, which is recognized by a host cell to terminate transcription. The terminator is operably linked to the 3′-terminus of the polynucleotide encoding the polypeptide. Any terminator that is functional in the host cell may be used in the present invention.

Preferred terminators for bacterial host cells are obtained from the genes for Bacillus clausii alkaline protease (aprH), Bacillus licheniformis alpha-amylase (amyL), and Escherichia coli ribosomal RNA (rmB).

Preferred terminators for filamentous fungal host cells are obtained from the genes for Aspergillus nidulans acetamidase, Aspergillus nidulans anthranilate synthase, Aspergillus niger glucoamylase, Aspergillus niger alpha-glucosidase, Aspergillus oryzae TAKA amylase, Fusarium oxysporum trypsin-like protease, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase Ill, Trichoderma reesei endoglucanase V, Trichoderma reesei xylanase I, Trichoderma reesei xylanase II, Trichoderma reesei xylanase Ill, Trichoderma reesei beta-xylosidase, and Trichoderma reesei translation elongation factor.

Preferred terminators for yeast host cells are obtained from the genes for Saccharomyces cerevisiae enolase, Saccharomyces cerevisiae cytochrome C (CYC1), and Saccharomyces cerevisiae glyceraldehyde-3-phosphate dehydrogenase. Other useful terminators for yeast host cells are described by Romanos et al., 1992, supra.

The control sequence may also be an mRNA stabilizer region downstream of a promoter and upstream of the coding sequence of a gene which increases expression of the gene.

Examples of suitable mRNA stabilizer regions are obtained from a Bacillus thuringiensis cryIIIA gene (WO 94/25612) and a Bacillus subtilis SP82 gene (Hue et al., 1995, Journal of Bacteriology 177: 3465-3471).

The control sequence may also be a leader, a nontranslated region of an mRNA that is important for translation by the host cell. The leader is operably linked to the 5′-terminus of the polynucleotide encoding the polypeptide. Any leader that is functional in the host cell may be used.

Preferred leaders for filamentous fungal host cells are obtained from the genes for Aspergillus oryzae TAKA amylase and Aspergillus nidulans triose phosphate isomerase.

Suitable leaders for yeast host cells are obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP).

The control sequence may also be a polyadenylation sequence, a sequence operably linked to the 3′-terminus of the polynucleotide and, when transcribed, is recognized by the host cell as a signal to add polyadenosine residues to transcribed mRNA. Any polyadenylation sequence that is functional in the host cell may be used.

Preferred polyadenylation sequences for filamentous fungal host cells are obtained from the genes for Aspergillus nidulans anthranilate synthase, Aspergillus niger glucoamylase, Aspergillus niger alpha-glucosidase Aspergillus oryzae TAKA amylase, and Fusarium oxysporum trypsin-like protease.

Useful polyadenylation sequences for yeast host cells are described by Guo and Sherman, 1995, Mol. Cellular Biol. 15: 5983-5990.

The control sequence may also be a signal peptide coding region that encodes a signal peptide linked to the N-terminus of a polypeptide and directs the polypeptide into the cell's secretory pathway. The 5′-end of the coding sequence of the polynucleotide may inherently contain a signal peptide coding sequence naturally linked in translation reading frame with the segment of the coding sequence that encodes the polypeptide. Alternatively, the 5′-end of the coding sequence may contain a signal peptide coding sequence that is foreign to the coding sequence. A foreign signal peptide coding sequence may be required where the coding sequence does not naturally contain a signal peptide coding sequence. Alternatively, a foreign signal peptide coding sequence may simply replace the natural signal peptide coding sequence in order to enhance secretion of the polypeptide. However, any signal peptide coding sequence that directs the expressed polypeptide into the secretory pathway of a host cell may be used.

Effective signal peptide coding sequences for bacterial host cells are the signal peptide coding sequences obtained from the genes for Bacillus NCIB 11837 maltogenic amylase, Bacillus licheniformis subtilisin, Bacillus licheniformis beta-lactamase, Bacillus stearothermophilus alpha-amylase, Bacillus stearothermophilus neutral proteases (nprT, nprS, nprM), and Bacillus subtilis prsA. Further signal peptides are described by Simonen and Palva, 1993, Microbiological Reviews 57: 109-137.

Effective signal peptide coding sequences for filamentous fungal host cells are the signal peptide coding sequences obtained from the genes for Aspergillus niger neutral amylase, Aspergillus niger glucoamylase, Aspergillus oryzae TAKA amylase, Humicola insolens cellulase, Humicola insolens endoglucanase V, Humicola lanuginosa lipase, and Rhizomucor miehei aspartic proteinase.

Useful signal peptides for yeast host cells are obtained from the genes for Saccharomyces cerevisiae alpha-factor and Saccharomyces cerevisiae invertase. Other useful signal peptide coding sequences are described by Romanos et al., 1992, supra.

The control sequence may also be a propeptide coding sequence that encodes a propeptide positioned at the N-terminus of a polypeptide. The resultant polypeptide is known as a proenzyme or propolypeptide (or a zymogen in some cases). A propolypeptide is generally inactive and can be converted to an active polypeptide by catalytic or autocatalytic cleavage of the propeptide from the propolypeptide. The propeptide coding sequence may be obtained from the genes for Bacillus subtilis alkaline protease (aprE), Bacillus subtilis neutral protease (nprT), Myceliophthora thermophila laccase (WO 95/33836), Rhizomucor miehei aspartic proteinase, and Saccharomyces cerevisiae alpha-factor.

Where both signal peptide and propeptide sequences are present, the propeptide sequence is positioned next to the N-terminus of a polypeptide and the signal peptide sequence is positioned next to the N-terminus of the propeptide sequence.

It may also be desirable to add regulatory sequences that regulate expression of the polypeptide relative to the growth of the host cell. Examples of regulatory sequences are those that cause expression of the gene to be turned on or off in response to a chemical or physical stimulus, including the presence of a regulatory compound. Regulatory sequences in prokaryotic systems include the lac, tac, and trp operator systems. In yeast, the ADH2 system or GAL1 system may be used. In filamentous fungi, the Aspergillus niger glucoamylase promoter, Aspergillus oryzae TAKA alpha-amylase promoter, and Aspergillus oryzae glucoamylase promoter, Trichoderma reesei cellobiohydrolase I promoter, and Trichoderma reesei cellobiohydrolase II promoter may be used. Other examples of regulatory sequences are those that allow for gene amplification. In eukaryotic systems, these regulatory sequences include the dihydrofolate reductase gene that is amplified in the presence of methotrexate, and the metallothionein genes that are amplified with heavy metals. In these cases, the polynucleotide encoding the polypeptide would be operably linked to the regulatory sequence.

### Expression Vectors

The present invention also relates to recombinant expression vectors comprising a polynucleotide of the present invention, a promoter, and transcriptional and translational stop signals. The various nucleotide and control sequences may be joined together to produce a recombinant expression vector that may include one or more convenient restriction sites to allow for insertion or substitution of the polynucleotide encoding the polypeptide at such sites. Alternatively, the polynucleotide may be expressed by inserting the polynucleotide or a nucleic acid construct comprising the polynucleotide into an appropriate vector for expression. In creating the expression vector, the coding sequence is located in the vector so that the coding sequence is operably linked with the appropriate control sequences for expression.

The recombinant expression vector may be any vector (e.g., a plasmid or virus) that can be conveniently subjected to recombinant DNA procedures and can bring about expression of the polynucleotide. The choice of the vector will typically depend on the compatibility of the vector with the host cell into which the vector is to be introduced. The vector may be a linear or closed circular plasmid.

The vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one that, when introduced into the host cell, is integrated into the genome and replicated together with the chromosome(s) into which it has been integrated. Furthermore, a single vector or plasmid or two or more vectors or plasmids that together contain the total DNA to be introduced into the genome of the host cell, or a transposon, may be used.

The vector preferably contains one or more selectable markers that permit easy selection of transformed, transfected, transduced, or the like cells. A selectable marker is a gene the product of which provides for biocide or viral resistance, resistance to heavy metals, prototrophy to auxotrophs, and the like.

Examples of bacterial selectable markers are Bacillus licheniformis or Bacillus subtilis dal genes, or markers that confer antibiotic resistance such as ampicillin, chloramphenicol, kanamycin, neomycin, spectinomycin, or tetracycline resistance. Suitable markers for yeast host cells include, but are not limited to, ADE2, HIS3, LEU2, LYS2, MET3, TRP1, and URA3. Selectable markers for use in a filamentous fungal host cell include, but are not limited to, adeA (phosphoribosylaminoimidazole-succinocarboxamide synthase), adeB (phosphoribosyl-aminoimidazole synthase), amdS (acetamidase), argB (ornithine carbamoyltransferase), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof. Preferred for use in an Aspergillus cell are Aspergillus nidulans or Aspergillus oryzae amdS and pyrG genes and a Streptomyces hygroscopicus bar gene. Preferred for use in a Trichoderma cell are adeA, adeB, amdS, hph, and pyrG genes.

The selectable marker may be a dual selectable marker system as described in WO 2010/039889. In one aspect, the dual selectable marker is an hph-tk dual selectable marker system.

The vector preferably contains an element(s) that permits integration of the vector into the host cell's genome or autonomous replication of the vector in the cell independent of the genome.

For integration into the host cell genome, the vector may rely on the polynucleotide's sequence encoding the polypeptide or any other element of the vector for integration into the genome by homologous or non-homologous recombination. Alternatively, the vector may contain additional polynucleotides for directing integration by homologous recombination into the genome of the host cell at a precise location(s) in the chromosome(s). To increase the likelihood of integration at a precise location, the integrational elements should contain a sufficient number of nucleic acids, such as 100 to 10,000 base pairs, 400 to 10,000 base pairs, and 800 to 10,000 base pairs, which have a high degree of sequence identity to the corresponding target sequence to enhance the probability of homologous recombination. The integrational elements may be any sequence that is homologous with the target sequence in the genome of the host cell. Furthermore, the integrational elements may be non-encoding or encoding polynucleotides. On the other hand, the vector may be integrated into the genome of the host cell by non-homologous recombination.

For autonomous replication, the vector may further comprise an origin of replication enabling the vector to replicate autonomously in the host cell in question. The origin of replication may be any plasmid replicator mediating autonomous replication that functions in a cell. The term “origin of replication” or “plasmid replicator” means a polynucleotide that enables a plasmid or vector to replicate in vivo.

Examples of bacterial origins of replication are the origins of replication of plasmids pBR322, pUC19, pACYC177, and pACYC184 permitting replication in E. coli, and pUB110, pE194, pTA1060, and pAMR1 permitting replication in Bacillus.

Examples of origins of replication for use in a yeast host cell are the 2 micron origin of replication, ARS1, ARS4, the combination of ARS1 and CEN3, and the combination of ARS4 and CEN6.

Examples of origins of replication useful in a filamentous fungal cell are AMA1 and ANS1 (Gems et al., 1991, Gene 98: 61-67; Cullen et al., 1987, Nucleic Acids Res. 15: 9163-9175; WO 00/24883). Isolation of the AMA1 gene and construction of plasmids or vectors comprising the gene can be accomplished according to the methods disclosed in WO 00/24883.

More than one copy of a polynucleotide of the present invention may be inserted into a host cell to increase production of a polypeptide. An increase in the copy number of the polynucleotide can be obtained by integrating at least one additional copy of the sequence into the host cell genome or by including an amplifiable selectable marker gene with the polynucleotide where cells containing amplified copies of the selectable marker gene, and thereby additional copies of the polynucleotide, can be selected for by cultivating the cells in the presence of the appropriate selectable agent.

The procedures used to ligate the elements described above to construct the recombinant expression vectors of the present invention are well known to one skilled in the art (see, e.g., Sambrook et al., 1989, supra).

### Host Cells

The present invention also relates to recombinant host cells, comprising a polynucleotide of the present invention operably linked to one or more control sequences that direct the production of a polypeptide of the present invention. A construct or vector comprising a polynucleotide is introduced into a host cell so that the construct or vector is maintained as a chromosomal integrant or as a self-replicating extra-chromosomal vector as described earlier. The term “host cell” encompasses any progeny of a parent cell that is not identical to the parent cell due to mutations that occur during replication. The choice of a host cell will to a large extent depend upon the gene encoding the polypeptide and its source.

The host cell may be any cell useful in the recombinant production of a polypeptide of the present invention, e.g., a prokaryote or a eukaryote.

The prokaryotic host cell may be any Gram-positive or Gram-negative bacterium. Gram-positive bacteria include, but are not limited to, Bacillus, Clostridium, Enterococcus, Geobacillus, Lactobacillus, Lactococcus, Oceanobacillus, Staphylococcus, Streptococcus, and Streptomyces. Gram-negative bacteria include, but are not limited to, Campylobacter, E. coli, Flavobacterium, Fusobacterium, Helicobacter, Ilyobacter, Neisseria, Pseudomonas, Salmonella, and Ureaplasma.

The bacterial host cell may be any Bacillus cell including, but not limited to, Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, and Bacillus thuringiensis cells.

The bacterial host cell may also be any Streptococcus cell including, but not limited to, Streptococcus equisimilis, Streptococcus pyogenes, Streptococcus uberis, and Streptococcus equi subsp. Zooepidemicus cells.

The bacterial host cell may also be any Streptomyces cell including, but not limited to, Streptomyces achromogenes, Streptomyces avermitilis, Streptomyces coelicolor, Streptomyces griseus, and Streptomyces lividans cells.

The introduction of DNA into a Bacillus cell may be effected by protoplast transformation (see, e.g., Chang and Cohen, 1979, Mol. Gen. Genet. 168: 111-115), competent cell transformation (see, e.g., Young and Spizizen, 1961, J. Bacteriol. 81: 823-829, or Dubnau and Davidoff-Abelson, 1971, J. Mol. Biol. 56: 209-221), electroporation (see, e.g., Shigekawa and Dower, 1988, Biotechniques 6: 742-751), or conjugation (see, e.g., Koehler and Thorne, 1987, J. Bacteriol. 169: 5271-5278). The introduction of DNA into an E. coli cell may be effected by protoplast transformation (see, e.g., Hanahan, 1983, J. Mol. Biol. 166: 557-580) or electroporation (see, e.g., Dower et al., 1988, Nucleic Acids Res. 16: 6127-6145). The introduction of DNA into a Streptomyces cell may be effected by protoplast transformation, electroporation (see, e.g., Gong et al., 2004, Folia Microbiol. (Praha) 49: 399-405), conjugation (see, e.g., Mazodier et al., 1989, J. Bacteriol. 171: 3583-3585), or transduction (see, e.g., Burke et al., 2001, Proc. Natl. Acad. Sci. USA 98: 6289-6294). The introduction of DNA into a Pseudomonas cell may be effected by electroporation (see, e.g., Choi et al., 2006, J. Microbiol. Methods 64: 391-397) or conjugation (see, e.g., Pinedo and Smets, 2005, Appl. Environ. Microbiol. 71: 51-57). The introduction of DNA into a Streptococcus cell may be effected by natural competence (see, e.g., Perry and Kuramitsu, 1981, Infect. Immun. 32: 1295-1297), protoplast transformation (see, e.g., Catt and Jollick, 1991, Microbios 68: 189-207), electroporation (see, e.g., Buckley et al., 1999, Appl. Environ. Microbiol. 65: 3800-3804), or conjugation (see, e.g., Clewell, 1981, Microbiol. Rev. 45: 409-436). However, any method known in the art for introducing DNA into a host cell can be used.

The host cell may also be a eukaryote, such as a mammalian, insect, plant, or fungal cell.

The host cell may be a fungal cell. “Fungi” as used herein includes the phyla Ascomycota, Basidiomycota, Chytridiomycota, and Zygomycota as well as the Oomycota and all mitosporic fungi (as defined by Hawksworth et al., In, Ainsworth and Bisby's Dictionary of The Fungi, 8th edition, 1995, CAB International, University Press, Cambridge, UK).

The fungal host cell may be a yeast cell. “Yeast” as used herein includes ascosporogenous yeast (Endomycetales), basidiosporogenous yeast, and yeast belonging to the Fungi Imperfecti (Blastomycetes). Since the classification of yeast may change in the future, for the purposes of this invention, yeast shall be defined as described in Biology and Activities of Yeast (Skinner, Passmore, and Davenport, editors, Soc. App. Bacteriol. Symposium Series No. 9, 1980).

The yeast host cell may be a Candida, Hansenula, Kluyveromyces, Pichia, Saccharomyces, Schizosaccharomyces, or Yarrowia cell, such as a Kluyveromyces lactis, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, or Yarrowia lipolytica cell.

The fungal host cell may be a filamentous fungal cell. “Filamentous fungi” include all filamentous forms of the subdivision Eumycota and Oomycota (as defined by Hawksworth et al., 1995, supra). The filamentous fungi are generally characterized by a mycelial wall composed of chitin, cellulose, glucan, chitosan, mannan, and other complex polysaccharides. Vegetative growth is by hyphal elongation and carbon catabolism is obligately aerobic. In contrast, vegetative growth by yeasts such as Saccharomyces cerevisiae is by budding of a unicellular thallus and carbon catabolism may be fermentative.

The filamentous fungal host cell may be an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell.

For example, the filamentous fungal host cell may be an Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma koningii, Trichoderma longibrachiatum, Trichoderma reesei, or Trichoderma viride cell.

Fungal cells may be transformed by a process involving protoplast formation, transformation of the protoplasts, and regeneration of the cell wall in a manner known per se. Suitable procedures for transformation of Aspergillus and Trichoderma host cells are described in EP 238023, Yelton et al., 1984, Proc. Natl. Acad. Sci. USA 81: 1470-1474, and Christensen et al., 1988, Bio/Technology 6: 1419-1422. Suitable methods for transforming Fusarium species are described by Malardier et al., 1989, Gene 78: 147-156, and WO 96/00787. Yeast may be transformed using the procedures described by Becker and Guarente, In Abelson, J. N. and Simon, M. I., editors, Guide to Yeast Genetics and Molecular Biology, Methods in Enzymology, Volume 194, pp 182-187, Academic Press, Inc., New York; Ito et al., 1983, J. Bacteriol. 153: 163; and Hinnen et al., 1978, Proc. Natl. Acad. Sci. USA 75: 1920.

### Methods of Production

The present invention also relates to methods of producing a polypeptide of the present invention, comprising (a) cultivating a cell, which in its wild-type form produces the polypeptide, under conditions conducive for production of the polypeptide; and optionally, (b) recovering the polypeptide. In one aspect, the cell is a Tenacibaculum cell. In another aspect, the cell is a Tenacibaculum geojense cell. In yet another aspect it is a Tenacibaculum sp-62066 cell. In another embodiment the cell is an Ahrensia cell, such as Ahrensia sp-62069. The present invention also relates to methods of producing a polypeptide of the present invention, comprising (a) cultivating a recombinant host cell of the present invention under conditions conducive for production of the polypeptide; and optionally, (b) recovering the polypeptide.

The host cells are cultivated in a nutrient medium suitable for production of the polypeptide using methods known in the art. For example, the cells may be cultivated by shake flask cultivation, or small-scale or large-scale fermentation (including continuous, batch, fed-batch, or solid state fermentations) in laboratory or industrial fermentors in a suitable medium and under conditions allowing the polypeptide to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). If the polypeptide is secreted into the nutrient medium, the polypeptide can be recovered directly from the medium. If the polypeptide is not secreted, it can be recovered from cell lysates.

The polypeptide may be detected using methods known in the art that are specific for the polypeptides. These detection methods include, but are not limited to, use of specific antibodies, formation of an enzyme product, or disappearance of an enzyme substrate. For example, an enzyme assay may be used to determine the activity of the polypeptide.

The polypeptide may be recovered using methods known in the art. For example, the polypeptide may be recovered from the nutrient medium by conventional procedures including, but not limited to, collection, centrifugation, filtration, extraction, spray-drying, evaporation, or precipitation. In one aspect, a fermentation broth comprising the polypeptide is recovered.

The polypeptide may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see, e.g., Protein Purification, Janson and Ryden, editors, VCH Publishers, New York, 1989) to obtain substantially pure polypeptides.

In an alternative aspect, the polypeptide is not recovered, but rather a host cell of the present invention expressing the polypeptide is used as a source of the polypeptide.

### Fermentation Broth Formulations or Cell Compositions

The present invention also relates to a fermentation broth formulation or a cell composition comprising a polypeptide of the present invention. The fermentation broth product further comprises additional ingredients used in the fermentation process, such as, for example, cells (including, the host cells containing the gene encoding the polypeptide of the present invention which are used to produce the polypeptide of interest), cell debris, biomass, fermentation media and/or fermentation products. In some embodiments, the composition is a cell-killed whole broth containing organic acid(s), killed cells and/or cell debris, and culture medium.

The term “fermentation broth” as used herein refers to a preparation produced by cellular fermentation that undergoes no or minimal recovery and/or purification. For example, fermentation broths are produced when microbial cultures are grown to saturation, incubated under carbon-limiting conditions to allow protein synthesis (e.g., expression of enzymes by host cells) and secretion into cell culture medium. The fermentation broth can contain unfractionated or fractionated contents of the fermentation materials derived at the end of the fermentation. Typically, the fermentation broth is unfractionated and comprises the spent culture medium and cell debris present after the microbial cells (e.g., filamentous fungal cells) are removed, e.g., by centrifugation. In some embodiments, the fermentation broth contains spent cell culture medium, extracellular enzymes, and viable and/or nonviable microbial cells.

In an embodiment, the fermentation broth formulation and cell compositions comprise a first organic acid component comprising at least one 1-5 carbon organic acid and/or a salt thereof and a second organic acid component comprising at least one 6 or more carbon organic acid and/or a salt thereof. In a specific embodiment, the first organic acid component is acetic acid, formic acid, propionic acid, a salt thereof, or a mixture of two or more of the foregoing and the second organic acid component is benzoic acid, cyclohexanecarboxylic acid, 4-methylvaleric acid, phenylacetic acid, a salt thereof, or a mixture of two or more of the foregoing.

In one aspect, the composition contains an organic acid(s), and optionally further contains killed cells and/or cell debris. In one embodiment, the killed cells and/or cell debris are removed from a cell-killed whole broth to provide a composition that is free of these components.

The fermentation broth formulations or cell compositions may further comprise a preservative and/or anti-microbial (e.g., bacteriostatic) agent, including, but not limited to, sorbitol, sodium chloride, potassium sorbate, and others known in the art.

The cell-killed whole broth or composition may contain the unfractionated contents of the fermentation materials derived at the end of the fermentation. Typically, the cell-killed whole broth or composition contains the spent culture medium and cell debris present after the microbial cells (e.g., filamentous fungal cells) are grown to saturation, incubated under carbon-limiting conditions to allow protein synthesis. In some embodiments, the cell-killed whole broth or composition contains the spent cell culture medium, extracellular enzymes, and killed filamentous fungal cells. In some embodiments, the microbial cells present in the cell-killed whole broth or composition can be permeabilized and/or lysed using methods known in the art.

A whole broth or cell composition as described herein is typically a liquid, but may contain insoluble components, such as killed cells, cell debris, culture media components, and/or insoluble enzyme(s). In some embodiments, insoluble components may be removed to provide a clarified liquid composition.

The whole broth formulations and cell compositions of the present invention may be produced by a method described in WO 90/15861 or WO 2010/096673.

### Enzyme Compositions

The present invention also relates to compositions comprising a polypeptide of the present invention. Preferably, the compositions are enriched in such a polypeptide. The term “enriched” indicates that the alpha-amylase activity of the composition has been increased, e.g., with an enrichment factor of at least 1.1.

The compositions may comprise a polypeptide of the present invention as the major enzymatic component, e.g., a mono-component composition. Alternatively, the compositions may comprise multiple enzymatic activities, such as one or more (e.g., several) enzymes selected from the group consisting of hydrolase, isomerase, ligase, lyase, oxidoreductase, or transferase, e.g., an alpha-galactosidase, alpha-glucosidase, aminopeptidase, amylase, beta-galactosidase, beta-glucosidase, beta-xylosidase, carbohydrase, carboxypeptidase, catalase, cellobiohydrolase, cellulase, chitinase, cutinase, cyclodextrin glycosyltransferase, deoxyribonuclease, endoglucanase, esterase, glucoamylase, invertase, laccase, lipase, mannosidase, mutanase, oxidase, pectinolytic enzyme, peroxidase, phytase, polyphenoloxidase, proteolytic enzyme, ribonuclease, transglutaminase, or xylanase.

The compositions may be prepared in accordance with methods known in the art and may be in the form of a liquid or a dry composition. The compositions may be stabilized in accordance with methods known in the art.

### Detergent Compositions

In one embodiment, the invention is directed to detergent compositions comprising an enzyme of the present invention in combination with one or more additional cleaning composition components. The choice of additional components is within the skill of the artisan and includes conventional ingredients, including the exemplary non-limiting components set forth below.

The choice of components may include, for textile care, the consideration of the type of textile to be cleaned, the type and/or degree of soiling, the temperature at which cleaning is to take place, and the formulation of the detergent product. Although components mentioned below are categorized by general header according to a particular functionality, this is not to be construed as a limitation, as a component may comprise additional functionalities as will be appreciated by the skilled artisan.

### Enzyme of the Present Invention

In one embodiment of the present invention, the a polypeptide of the present invention may be added to a detergent composition in an amount corresponding to 0.001-200 mg of protein, such as 0.005-100 mg of protein, preferably 0.01-50 mg of protein, more preferably 0.05-20 mg of protein, even more preferably 0.1-10 mg of protein per liter of wash liquor.

The enzyme(s) of the detergent composition of the invention may be stabilized using conventional stabilizing agents, e.g. a polyol such as propylene glycol or glycerol, a sugar or sugar alcohol, lactic acid, boric acid, or a boric acid derivative, e.g. an aromatic borate ester, or a phenyl boronic acid derivative such as 4-formylphenyl boronic acid, and the composition may be formulated as described in, for example, WO92/19709 and WO92/19708.

A polypeptide of the present invention may also be incorporated in the detergent formulations disclosed in WO97/07202, which is hereby incorporated by reference.

### Surfactants

The detergent composition may comprise one or more surfactants, which may be anionic and/or cationic and/or non-ionic and/or semi-polar and/or zwitterionic, or a mixture thereof. In a particular embodiment, the detergent composition includes a mixture of one or more nonionic surfactants and one or more anionic surfactants. The surfactant(s) is typically present at a level of from about 0.1% to 60% by weight, such as about 1% to about 40%, or about 3% to about 20%, or about 3% to about 10%. The surfactant(s) is chosen based on the desired cleaning application, and includes any conventional surfactant(s) known in the art. Any surfactant known in the art for use in detergents may be utilized.

When included therein the detergent will usually contain from about 1% to about 40% by weight, such as from about 5% to about 30%, including from about 5% to about 15%, or from about 20% to about 25% of an anionic surfactant. Non-limiting examples of anionic surfactants include sulfates and sulfonates, in particular, linear alkylbenzenesulfonates (LAS), branched alkylbenzenesulfonates (BABS), phenylalkanesulfonates, alpha-olefinsulfonates (AOS), olefin sulfonates, alkene sulfonates, alkane-2,3-diylbis(sulfates), hydroxyalkanesulfonates and disulfonates, alkyl sulfates (AS) such as sodium dodecyl sulfate (SDS), fatty alcohol sulfates (FAS), primary alcohol sulfates (PAS), alcohol ethersulfates (AES or AEOS or FES, also known as alcohol ethoxysulfates or fatty alcohol ether sulfates), secondary alkanesulfonates (SAS), paraffin sulfonates (PS), ester sulfonates, sulfonated fatty acid glycerol esters, alpha-sulfo fatty acid methyl esters (alpha-SFMe or SES) including methyl ester sulfonate (MES), alkyl- or alkenylsuccinic acid, dodecenyl/tetradecenyl succinic acid (DTSA), fatty acid derivatives of amino acids, diesters and monoesters of sulfo-succinic acid or soap, and combinations thereof.

When included therein the detergent will usually contain from about 1% to about 40% by weight of a cationic surfactant. Non-limiting examples of cationic surfactants include alklydimethylethanolamine quat (ADMEAQ), cetyltrimethylammonium bromide (CTAB), dimethyldistearylammonium chloride (DSDMAC), and alkylbenzyldimethylammonium, alkyl quaternary ammonium compounds, alkoxylated quaternary ammonium (AQA) compounds, and combinations thereof.

When included therein the detergent will usually contain from about 0.2% to about 40% by weight of a non-ionic surfactant, for example from about 0.5% to about 30%, in particular from about 1% to about 20%, from about 3% to about 10%, such as from about 3% to about 5%, or from about 8% to about 12%. Non-limiting examples of non-ionic surfactants include alcohol ethoxylates (AE or AEO), alcohol propoxylates, propoxylated fatty alcohols (PFA), alkoxylated fatty acid alkyl esters, such as ethoxylated and/or propoxylated fatty acid alkyl esters, alkylphenol ethoxylates (APE), nonylphenol ethoxylates (NPE), alkylpolyglycosides (APG), alkoxylated amines, fatty acid monoethanolamides (FAM), fatty acid diethanolamides (FADA), ethoxylated fatty acid monoethanolamides (EFAM), propoxylated fatty acid monoethanolamides (PFAM), polyhydroxy alkyl fatty acid amides, or N-acyl N-alkyl derivatives of glucosamine (glucamides, GA, or fatty acid glucamide, FAGA), as well as products available under the trade names SPAN and TWEEN, and combinations thereof.

When included therein the detergent will usually contain from about 1% to about 20% by weight of a semipolar surfactant. Non-limiting examples of semipolar surfactants include amine oxides (AO) such as alkyldimethylamineoxide, N-(coco alkyl)-N,N-dimethylamine oxide and N-(tallow-alkyl)-N,N-bis(2-hydroxyethyl)amine oxide, fatty acid alkanolamides and ethoxylated fatty acid alkanolamides, and combinations thereof.

When included therein the detergent will usually contain from about 1% to about 20% by weight of a zwitterionic surfactant. Non-limiting examples of zwitterionic surfactants include betaine, alkyldimethylbetaine, sulfobetaine, and combinations thereof.

### Hydrotropes

A hydrotrope is a compound that solubilises hydrophobic compounds in aqueous solutions (or oppositely, polar substances in a non-polar environment). Typically, hydrotropes have both hydrophilic and a hydrophobic character (so-called amphiphilic properties as known from surfactants); however the molecular structure of hydrotropes generally do not favor spontaneous self-aggregation, see e.g. review by Hodgdon and Kaler (2007), Current Opinion in Colloid & Interface Science 12: 121-128. Hydrotropes do not display a critical concentration above which self-aggregation occurs as found for surfactants and lipids forming miceller, lamellar or other well defined meso-phases. Instead, many hydrotropes show a continuous-type aggregation process where the sizes of aggregates grow as concentration increases. However, many hydrotropes alter the phase behavior, stability, and colloidal properties of systems containing substances of polar and non-polar character, including mixtures of water, oil, surfactants, and polymers. Hydrotropes are classically used across industries from pharma, personal care, food, to technical applications. Use of hydrotropes in detergent compositions allow for example more concentrated formulations of surfactants (as in the process of compacting liquid detergents by removing water) without inducing undesired phenomena such as phase separation or high viscosity.

The detergent may contain 0-5% by weight, such as about 0.5 to about 5%, or about 3% to about 5%, of a hydrotrope. Any hydrotrope known in the art for use in detergents may be utilized. Non-limiting examples of hydrotropes include sodium benzene sulfonate, sodium p-toluene sulfonate (STS), sodium xylene sulfonate (SXS), sodium cumene sulfonate (SCS), sodium cymene sulfonate, amine oxides, alcohols and polyglycolethers, sodium hydroxynaphthoate, sodium hydroxynaphthalene sulfonate, sodium ethylhexyl sulfate, and combinations thereof.

### Builders and Co-Builders

The detergent composition may contain about 0-65% by weight, such as about 5% to about 40% of a detergent builder or co-builder, or a mixture thereof. In a dish wash deteregent, the level of builder is typically 40-65%, particularly 50-65%. The builder and/or co-builder may particularly be a chelating agent that forms water-soluble complexes with Ca and Mg. Any builder and/or co-builder known in the art for use in laundry detergents may be utilized. Non-limiting examples of builders include zeolites, diphosphates (pyrophosphates), triphosphates such as sodium triphosphate (STP or STPP), carbonates such as sodium carbonate, soluble silicates such as sodium metasilicate, layered silicates (e.g., SKS-6 from Hoechst), ethanolamines such as 2-aminoethan-1-ol (MEA), diethanolamine (DEA, also known as iminodiethanol), triethanolamine (TEA, also known as 2,2′,2″-nitrilotriethanol), and carboxymethyl inulin (CMI), and combinations thereof.

The detergent composition may also contain 0-40% by weight, such as about 5% to about 20%, of a detergent co-builder, or a mixture thereof. The detergent composition may include include a co-builder alone, or in combination with a builder, for example a zeolite builder. Non-limiting examples of co-builders include homopolymers of polyacrylates or copolymers thereof, such as poly(acrylic acid) (PAA) or copoly(acrylic acid/maleic acid) (PAA/PMA). Further non-limiting examples include citrate, chelators such as aminocarboxylates, aminopolycarboxylates and phosphonates, and alkyl- or alkenylsuccinic acid. Additional specific examples include 2,2′,2″-nitrilotriacetic acid (NTA), ethylenediaminetetraacetic acid (EDTA), diethylenetriaminepentaacetic acid (DTPA), iminodisuccinic acid (IDS), ethylenediamine-N,N′-disuccinic acid (EDDS), methylglycinediacetic acid (MGDA), glutamic acid-N,N-diacetic acid (GLDA), 1-hydroxyethane-1,1-diphosphonic acid (HEDP), ethylenediaminetetra(methylenephosphonic acid) (EDTMPA), diethylenetriaminepentakis(methylenephosphonic acid) (DTMPA or DTPMPA), N-(2-hydroxyethyl)iminodiacetic acid (EDG), aspartic acid-N-monoacetic acid (ASMA), aspartic acid-N,N-diacetic acid (ASDA), aspartic acid-N-monopropionic acid (ASMP), iminodisuccinic acid (IDA), N-(2-sulfomethyl)-aspartic acid (SMAS), N-(2-sulfoethyl)-aspartic acid (SEAS), N-(2-sulfomethyl)-glutamic acid (SMGL), N-(2-sulfoethyl)-glutamic acid (SEGL), N-methyliminodiacetic acid (MIDA), α-alanine-N, N-diacetic acid (α-ALDA), serine-N, N-diacetic acid (SEDA), isoserine-N, N-diacetic acid (ISDA), phenylalanine-N, N-diacetic acid (PHDA), anthranilic acid-N, N-diacetic acid (ANDA), sulfanilic acid-N, N-diacetic acid (SLDA), taurine-N, N-diacetic acid (TUDA) and sulfomethyl-N, N-diacetic acid (SMDA), N-(2-hydroxyethyl)-ethylidenediamine-N, N′, N′-triacetate (HEDTA), diethanolglycine (DEG), diethylenetriamine penta(methylenephosphonic acid) (DTPMP), aminotris(methylenephosphonic acid) (ATMP), and combinations and salts thereof. Further exemplary builders and/or co-builders are described in, e.g., WO 09/102854, U.S. Pat. No. 5,977,053.

### Bleaching Systems

The detergent may contain 0-50% by weight, such as about 5% to about 40%, of a bleaching system. Any bleaching system known in the art for use in laundry detergents may be utilized. Suitable bleaching system components include bleaching catalysts, photobleaches, bleach activators, sources of hydrogen peroxide such as sodium percarbonate and sodium perborates, preformed peracids and mixtures thereof. Suitable preformed peracids include, but are not limited to, peroxycarboxylic acids and salts, percarbonic acids and salts, perimidic acids and salts, peroxymonosulfuric acids and salts, for example, Oxone (R), and mixtures thereof. Non-limiting examples of bleaching systems include peroxide-based bleaching systems, which may comprise, for example, an inorganic salt, including alkali metal salts such as sodium salts of perborate (usually mono- or tetra-hydrate), percarbonate, persulfate, perphosphate, persilicate salts, in combination with a peracid-forming bleach activator. The term bleach activator is meant herein as a compound which reacts with peroxygen bleach like hydrogen peroxide to form a peracid. The peracid thus formed constitutes the activated bleach. Suitable bleach activators to be used herein include those belonging to the class of esters amides, imides or anhydrides. Suitable examples are tetracetylethylene diamine (TAED), sodium 4-[(3,5,5-trimethylhexanoyl)oxy]benzene sulfonate (ISONOBS), diperoxy dodecanoic acid, 4-(dodecanoyloxy)benzenesulfonate (LOBS), 4-(decanoyloxy)benzenesulfonate, 4-(decanoyloxy)benzoate (DOBS), 4-(nonanoyloxy)-benzenesulfonate (NOBS), and/or those disclosed in WO98/17767. A particular family of bleach activators of interest was disclosed in EP624154 and particulary preferred in that family is acetyl triethyl citrate (ATC). ATC or a short chain triglyceride like triacetin has the advantage that it is environmental friendly as it eventually degrades into citric acid and alcohol. Furthermore acetyl triethyl citrate and triacetin has a good hydrolytical stability in the product upon storage and it is an efficient bleach activator. Finally ATC provides a good building capacity to the laundry additive. Alternatively, the bleaching system may comprise peroxyacids of, for example, the amide, imide, or sulfone type. The bleaching system may also comprise peracids such as 6-(phthalimido)peroxyhexanoic acid (PAP). The bleaching system may also include a bleach catalyst. In some embodiments the bleach component may be an organic catalyst selected from the group consisting of organic catalysts having the following structures:

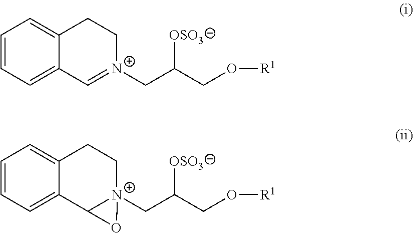

(iii) and mixtures thereof; wherein each R¹ is independently a branched alkyl group containing from 9 to 24 carbons or linear alkyl group containing from 11 to 24 carbons, preferably each R¹ is independently a branched alkyl group containing from 9 to 18 carbons or linear alkyl group containing from 11 to 18 carbons, more preferably each R¹ is independently selected from the group consisting of 2-propylheptyl, 2-butyloctyl, 2-pentylnonyl, 2-hexyldecyl, n-dodecyl, n-tetradecyl, n-hexadecyl, n-octadecyl, iso-nonyl, iso-decyl, iso-tridecyl and iso-pentadecyl. Other exemplary bleaching systems are described, e.g. in WO2007/087258, WO2007/087244, WO2007/087259 and WO2007/087242. Suitable photobleaches may for example be sulfonated zinc phthalocyanine

### Polymers

The detergent may contain 0-10% by weight, such as 0.5-5%, 2-5%, 0.5-2% or 0.2-1% of a polymer. Any polymer known in the art for use in detergents may be utilized. The polymer may function as a co-builder as mentioned above, or may provide antiredeposition, fiber protection, soil release, dye transfer inhibition, grease cleaning and/or anti-foaming properties. Some polymers may have more than one of the above-mentioned properties and/or more than one of the below-mentioned motifs. Exemplary polymers include (carboxymethyl)cellulose (CMC), poly(vinyl alcohol) (PVA), poly(vinylpyrrolidone) (PVP), poly(ethyleneglycol) or poly(ethylene oxide) (PEG), ethoxylated poly(ethyleneimine), carboxymethyl inulin (CMI), and polycarboxylates such as PAA, PAA/PMA, poly-aspartic acid, and lauryl methacrylate/acrylic acid copolymers, hydrophobically modified CMC (HM-CMC) and silicones, copolymers of terephthalic acid and oligomeric glycols, copolymers of poly(ethylene terephthalate) and poly(oxyethene terephthalate) (PET-POET), PVP, poly(vinylimidazole) (PVI), poly(vinylpyridine-N-oxide) (PVPO or PVPNO) and polyvinylpyrrolidone-vinylimidazole (PVPVI). Further exemplary polymers include sulfonated polycarboxylates, polyethylene oxide and polypropylene oxide (PEO-PPO) and diquaternium ethoxy sulfate. Other exemplary polymers are disclosed in, e.g., WO 2006/130575. Salts of the above-mentioned polymers are also contemplated.

### Fabric Hueing Agents

The detergent compositions of the present invention may also include fabric hueing agents such as dyes or pigments, which when formulated in detergent compositions can deposit onto a fabric when said fabric is contacted with a wash liquor comprising said detergent compositions and thus altering the tint of said fabric through absorption/reflection of visible light. Fluorescent whitening agents emit at least some visible light. In contrast, fabric hueing agents alter the tint of a surface as they absorb at least a portion of the visible light spectrum. Suitable fabric hueing agents include dyes and dye-clay conjugates, and may also include pigments. Suitable dyes include small molecule dyes and polymeric dyes. Suitable small molecule dyes include small molecule dyes selected from the group consisting of dyes falling into the Colour Index (C.I.) classifications of Direct Blue, Direct Red, Direct Violet, Acid Blue, Acid Red, Acid Violet, Basic Blue, Basic Violet and Basic Red, or mixtures thereof, for example as described in WO2005/03274, WO2005/03275, WO2005/03276 and EP1876226 (hereby incorporated by reference). The detergent composition preferably comprises from about 0.00003 wt % to about 0.2 wt %, from about 0.00008 wt % to about 0.05 wt %, or even from about 0.0001 wt % to about 0.04 wt % fabric hueing agent. The composition may comprise from 0.0001 wt % to 0.2 wt % fabric hueing agent, this may be especially preferred when the composition is in the form of a unit dose pouch. Suitable hueing agents are also disclosed in, e.g. WO 2007/087257 and WO2007/087243.

### (Additional) Enzymes

The detergent additive as well as the detergent composition may comprise one or more additional enzymes such as a protease, lipase, cutinase, an amylase, carbohydrase, cellulase, pectinase, mannanase, arabinase, galactanase, xylanase, oxidase, e.g., a laccase, and/or peroxidase.

In general the properties of the selected enzyme(s) should be compatible with the selected detergent, (i.e., pH-optimum, compatibility with other enzymatic and non-enzymatic ingredients, etc.), and the enzyme(s) should be present in effective amounts.

Cellulases: Suitable cellulases include those of bacterial or fungal origin. Chemically modified or protein engineered mutants are included. Suitable cellulases include cellulases from the genera Bacillus, Pseudomonas, Humicola, Fusarium, Thielavia, Acremonium, e.g., the fungal cellulases produced from Humicola insolens, Myceliophthora thermophila and Fusarium oxysporum disclosed in U.S. Pat. No. 4,435,307, U.S. Pat. No. 5,648,263, U.S. Pat. No. 5,691,178, U.S. Pat. No. 5,776,757 and WO 89/09259.

Especially suitable cellulases are the alkaline or neutral cellulases having colour care benefits. Examples of such cellulases are cellulases described in EP 0 495 257, EP 0 531 372, WO 96/11262, WO 96/29397, WO 98/08940. Other examples are cellulase variants such as those described in WO 94/07998, EP 0 531 315, U.S. Pat. No. 5,457,046, U.S. Pat. No. 5,686,593, U.S. Pat. No. 5,763,254, WO 95/24471, WO 98/12307 and PCT/DK98/00299.

Commercially available cellulases include Celluzyme™, and Carezyme™ (Novozymes A/S), Clazinase™, and Puradax HA™ (Genencor International Inc.), and KAC-500(B)™ (Kao Corporation).

Proteases: Suitable proteases include those of animal, vegetable or microbial origin. Microbial origin is preferred. Chemically modified or protein engineered mutants are included. The protease may be a serine protease or a metalloprotease, preferably an alkaline microbial protease or a trypsin-like protease. Examples of alkaline proteases are subtilisins, especially those derived from Bacillus, e.g., subtilisin Novo, subtilisin Carlsberg, subtilisin 309, subtilisin 147 and subtilisin 168 (described in WO 89/06279). Examples of trypsin-like proteases are trypsin (e.g., of porcine or bovine origin) and the Fusarium protease described in WO 89/06270 and WO 94/25583.

Examples of useful proteases are the variants described in WO 92/19729, WO 98/20115, WO 98/20116, and WO 98/34946, especially the variants with substitutions in one or more of the following positions: 27, 36, 57, 76, 87, 97, 101, 104, 120, 123, 167, 170, 194, 206, 218, 222, 224, 235, and 274.

Preferred commercially available protease enzymes include Alcalase™, Savinase™ Primase™, Duralase™, Esperase™, and Kannase™ (Novozymes NS), Maxatase™, Maxacal™ Maxapem™, Properase™, Purafect™, Purafect OxP™, FN2™, and FN3™ (Genencor International Inc.).

Lipases and Cutinases: Suitable lipases and cutinases include those of bacterial or fungal origin. Chemically modified or protein engineered mutant enzymes are included. Examples include lipase from Thermomyces, e.g. from T. lanuginosus (previously named Humicola lanuginosa) as described in EP258068 and EP305216, cutinase from Humicola, e.g. H. insolens (WO96/13580), lipase from strains of Pseudomonas (some of these now renamed to Burkholderia), e.g. P. alcaligenes or P. pseudoalcaligenes (EP218272), P. cepacia (EP331376), P. sp. strain SD705 (WO95/06720 & WO96/27002), P. wisconsinensis (WO96/12012), GDSL-type Streptomyces lipases (WO10/065455), cutinase from Magnaporthe grisea (WO10/107560), cutinase from Pseudomonas mendocina (U.S. Pat. No. 5,389,536), lipase from Thermobifida fusca (WO11/084412), Geobacillus stearothermophilus lipase (WO11/084417), lipase from Bacillus subtilis (WO11/084599), and lipase from Streptomyces griseus (WO11/150157) and S. pristinaespiralis (WO12/137147).

Other examples are lipase variants such as those described in EP407225, WO92/05249, WO94/01541, WO94/25578, WO95/14783, WO95/30744, WO95/35381, WO95/22615, WO96/00292, WO97/04079, WO97/07202, WO00/34450, WO00/60063, WO01/92502, WO07/87508 and WO09/109500.

Preferred commercial lipase products include include Lipolase™, Lipex™; Lipolex™ and Lipoclean™ (Novozymes A/S), Lumafast (originally from Genencor) and Lipomax (originally from Gist-Brocades).

Still other examples are lipases sometimes referred to as acyltransferases or perhydrolases, e.g. acyltransferases with homology to Candida antarctica lipase A (WO10/111143), acyltransferase from Mycobacterium smegmatis (WO05/56782), perhydrolases from the CE 7 family (WO09/67279), and variants of the M. smegmatis perhydrolase in particular the S54V variant used in the commercial product Gentle Power Bleach from Huntsman Textile Effects Pte Ltd (WO10/100028).

Amylases: Suitable amylases which can be used together with the enzyme of the invention may be an alpha-amylase or a glucoamylase and may be of bacterial or fungal origin. Chemically modified or protein engineered mutants are included. Amylases include, for example, alpha-amylases obtained from Bacillus, e.g., a special strain of Bacillus licheniformis, described in more detail in GB 1,296,839.

Suitable amylases include amylases having SEQ ID NO: 3 in WO 95/10603 or variants having 90% sequence identity to SEQ ID NO: 3 thereof. Preferred variants are described in WO 94/02597, WO 94/18314, WO 97/43424 and SEQ ID NO: 4 of WO 99/019467, such as variants with substitutions in one or more of the following positions: 15, 23, 105, 106, 124, 128, 133, 154, 156, 178, 179, 181, 188, 190, 197, 201, 202, 207, 208, 209, 211, 243, 264, 304, 305, 391, 408, and 444.

Different suitable amylases include amylases having SEQ ID NO: 6 in WO 02/010355 or variants thereof having 90% sequence identity to SEQ ID NO: 6. Preferred variants of SEQ ID NO: 6 are those having a deletion in positions 181 and 182 and a substitution in position 193.

Other amylases which are suitable are hybrid alpha-amylase comprising residues 1-33 of the alpha-amylase derived from B. amyloliquefaciens shown in SEQ ID NO: 6 of WO 2006/066594 and residues 36-483 of the B. licheniformis alpha-amylase shown in SEQ ID NO: 4 of WO 2006/066594 or variants having 90% sequence identity thereof. Preferred variants of this hybrid alpha-amylase are those having a substitution, a deletion or an insertion in one of more of the following positions: G48, T49, G107, H156, A181, N190, M197, I201, A209 and Q264. Most preferred variants of the hybrid alpha-amylase comprising residues 1-33 of the alpha-amylase derived from B. amyloliquefaciens shown in SEQ ID NO: 6 of WO 2006/066594 and residues 36-483 of SEQ ID NO: 4 are those having the substitutions:

M197T;

H156Y+A181T+N190F+A209V+Q264S; or

G48A+T49I+G107A+H156Y+A181T+N190F+I201F+A209V+Q264S.

Further amylases which are suitable are amylases having SEQ ID NO: 6 in WO 99/019467 or variants thereof having 90% sequence identity to SEQ ID NO: 6. Preferred variants of SEQ ID NO: 6 are those having a substitution, a deletion or an insertion in one or more of the following positions: R181, G182, H183, G184, N195, I206, E212, E216 and K269. Particularly preferred amylases are those having deletion in positions R181 and G182, or positions H183 and G184.

Additional amylases which can be used are those having SEQ ID NO: 1, SEQ ID NO: 3, SEQ ID NO: 2 or SEQ ID NO: 7 of WO 96/023873 or variants thereof having 90% sequence identity to SEQ ID NO: 1, SEQ ID NO: 2, SEQ ID NO: 3 or SEQ ID NO: 7. Preferred variants of SEQ ID NO: 1, SEQ ID NO: 2, SEQ ID NO: 3 or SEQ ID NO: 7 are those having a substitution, a deletion or an insertion in one or more of the following positions: 140, 181, 182, 183, 184, 195, 206, 212, 243, 260, 269, 304 and 476. More preferred variants are those having a deletion in positions 181 and 182 or positions 183 and 184. Most preferred amylase variants of SEQ ID NO: 1, SEQ ID NO: 2 or SEQ ID NO: 7 are those having a deletion in positions 183 and 184 and a substitution in one or more of positions 140, 195, 206, 243, 260, 304 and 476.

Other amylases which can be used are amylases having SEQ ID NO: 2 of WO 08/153815, SEQ ID NO: 10 in WO 01/66712 or variants thereof having 90% sequence identity to SEQ ID NO: 2 of WO 08/153815 or 90% sequence identity to SEQ ID NO: 10 in WO 01/66712. Preferred variants of SEQ ID NO: 10 in WO 01/66712 are those having a substitution, a deletion or an insertion in one of more of the following positions: 176, 177, 178, 179, 190, 201, 207, 211 and 264.

Further suitable amylases are amylases having SEQ ID NO: 2 of WO 09/061380 or variants having 90% sequence identity to SEQ ID NO: 2 thereof. Preferred variants of SEQ ID NO: 2 are those having a truncation of the C-terminus and/or a substitution, a deletion or an insertion in one of more of the following positions: Q87, Q98, S125, N128, T131, T165, K178, R180, S181, T182, G183, M201, F202, N225, S243, N272, N282, Y305, R309, D319, Q320, Q359, K444 and G475. More preferred variants of SEQ ID NO: 2 are those having the substitution in one of more of the following positions: Q87E,R, Q98R, S125A, N128C, T131I, T165I, K178L, T182G, M201L, F202Y, N225E,R, N272E,R, S243Q,A,E,D, Y305R, R309A, Q320R, Q359E, K444E and G475K and/or deletion in position R180 and/or S181 or of T182 and/or G183. Most preferred amylase variants of SEQ ID NO: 2 are those having the substitutions:

N128C+K178L+T182G+Y305R+G475K;

N128C+K178L+T182G+F202Y+Y305R+D319T+G475K;

S125A+N128C+K178L+T182G+Y305R+G475K; or

S125A+N128C+T131I+T165I+K178L+T182G+Y305R+G475K wherein the variants are C-terminally truncated and optionally further comprises a substitution at position 243 and/or a deletion at position 180 and/or position 181.

Other suitable amylases are the alpha-amylase having SEQ ID NO: 12 in WO01/66712 or a variant having at least 90% sequence identity to SEQ ID NO: 12. Preferred amylase variants are those having a substitution, a deletion or an insertion in one of more of the following positions of SEQ ID NO: 12 in WO01/66712: R28, R118, N174; R181, G182, D183, G184, G186, W189, N195, M202, Y298, N299, K302, S303, N306, R310, N314; R320, H324, E345, Y396, R400, W439, R444, N445, K446, Q449, R458, N471, N484. Particular preferred amylases include variants having a deletion of D183 and G184 and having the substitutions R118K, N195F, R320K and R458K, and a variant additionally having substitutions in one or more position selected from the group: M9, G149, G182, G186, M202, T257, Y295, N299, M323, E345 and A339, most preferred a variant that additionally has substitutions in all these positions.

Other examples are amylase variants such as those described in WO2011/098531, WO2013/001078 and WO2013/001087.

Commercially available amylases are Duramyl™, Termamyl™, Fungamyl™, Stainzyme™, Stainzyme Plus™, Natalase™, Liquozyme X and BAN™ (from Novozymes NS), and Rapidase™, Purastar™, and Powerase (from Genencor International Inc.).

Peroxidases/Oxidases: Suitable peroxidases/oxidases include those of plant, bacterial or fungal origin. Chemically modified or protein engineered mutants are included. Examples of useful peroxidases include peroxidases from Coprinus, e.g., from C. cinereus, and variants thereof as those described in WO 93/24618, WO 95/10602, and WO 98/15257.

Commercially available peroxidases include Guardzyme™ (Novozymes NS).

The detergent enzyme(s) may be included in a detergent composition by adding separate additives containing one or more enzymes, or by adding a combined additive comprising all of these enzymes. A detergent additive of the invention, i.e., a separate additive or a combined additive, can be formulated, for example, as a granulate, liquid, slurry, etc. Preferred detergent additive formulations are granulates, in particular non-dusting granulates, liquids, in particular stabilized liquids, or slurries.

Non-dusting granulates may be produced, e.g. as disclosed in U.S. Pat. Nos. 4,106,991 and 4,661,452 and may optionally be coated by methods known in the art. Examples of waxy coating materials are poly(ethylene oxide) products (polyethyleneglycol, PEG) with mean molar weights of 1000 to 20000; ethoxylated nonylphenols having from 16 to 50 ethylene oxide units; ethoxylated fatty alcohols in which the alcohol contains from 12 to 20 carbon atoms and in which there are 15 to 80 ethylene oxide units; fatty alcohols; fatty acids; and mono- and di- and triglycerides of fatty acids. Examples of film-forming coating materials suitable for application by fluid bed techniques are given in GB 1483591. Liquid enzyme preparations may, for instance, be stabilized by adding a polyol such as propylene glycol, a sugar or sugar alcohol, lactic acid or boric acid according to established methods. Protected enzymes may be prepared according to the method disclosed in EP 238,216.

### Adjunct Materials

Any detergent components known in the art for use in laundry detergents may also be utilized. Other optional detergent components include anti-corrosion agents, anti-shrink agents, anti-soil redeposition agents, anti-wrinkling agents, bactericides, binders, corrosion inhibitors, disintegrants/disintegration agents, dyes, enzyme stabilizers (including boric acid, borates, CMC, and/or polyols such as propylene glycol), fabric conditioners including clays, fillers/processing aids, fluorescent whitening agents/optical brighteners, foam boosters, foam (suds) regulators, perfumes, soil-suspending agents, softeners, suds suppressors, tarnish inhibitors, and wicking agents, either alone or in combination. Any ingredient known in the art for use in laundry detergents may be utilized. The choice of such ingredients is well within the skill of the artisan.

Dispersants—The detergent compositions of the present invention can also contain dispersants. In particular powdered detergents may comprise dispersants. Suitable water-soluble organic materials include the homo- or co-polymeric acids or their salts, in which the polycarboxylic acid comprises at least two carboxyl radicals separated from each other by not more than two carbon atoms. Suitable dispersants are for example described in Powdered Detergents, Surfactant science series volume 71, Marcel Dekker, Inc.

Dye Transfer Inhibiting Agents—The detergent compositions of the present invention may also include one or more dye transfer inhibiting agents. Suitable polymeric dye transfer inhibiting agents include, but are not limited to, polyvinylpyrrolidone polymers, polyamine N-oxide polymers, copolymers of N-vinylpyrrolidone and N-vinylimidazole, polyvinyloxazolidones and polyvinylimidazoles or mixtures thereof. When present in a subject composition, the dye transfer inhibiting agents may be present at levels from about 0.0001% to about 10%, from about 0.01% to about 5% or even from about 0.1% to about 3% by weight of the composition.

Fluorescent whitening agent—The detergent compositions of the present invention will preferably also contain additional components that may tint articles being cleaned, such as fluorescent whitening agent or optical brighteners. Where present the brightener is preferably at a level of about 0.01% to about 0.5%. Any fluorescent whitening agent suitable for use in a laundry detergent composition may be used in the composition of the present invention. The most commonly used fluorescent whitening agents are those belonging to the classes of diaminostilbene-sulfonic acid derivatives, diarylpyrazoline derivatives and bisphenyl-distyryl derivatives. Examples of the diaminostilbene-sulfonic acid derivative type of fluorescent whitening agents include the sodium salts of: 4,4′-bis-(2-diethanolamino-4-anilino-s-triazin-6-ylamino) stilbene-2,2′-disulfonate, 4,4′-bis-(2,4-dianilino-s-triazin-6-ylamino) stilbene-2,2′-disulfonate, 4,4′-bis-(2-anilino-4-(N-methyl-N-2-hydroxy-ethylamino)-s-triazin-6-ylamino) stilbene-2,2′-disulfonate, 4,4′-bis-(4-phenyl-1,2,3-triazol-2-yl)stilbene-2,2′-disulfonate and sodium 5-(2H-naphtho[1,2-d][1,2,3]triazol-2-yl)-2-[(E)-2-phenylvinyl]benzenesulfonate. Preferred fluorescent whitening agents are Tinopal DMS and Tinopal CBS available from Ciba-Geigy AG, Basel, Switzerland. Tinopal DMS is the disodium salt of 4,4′-bis-(2-morpholino-4-anilino-s-triazin-6-ylamino) stilbene-2,2′-disulfonate. Tinopal CBS is the disodium salt of 2,2′-bis-(phenyl-styryl)-disulfonate. Also preferred are fluorescent whitening agents is the commercially available Parawhite KX, supplied by Paramount Minerals and Chemicals, Mumbai, India. Other fluorescers suitable for use in the invention include the 1-3-diaryl pyrazolines and the 7-alkylaminocoumarins.

Suitable fluorescent brightener levels include lower levels of from about 0.01, from 0.05, from about 0.1 or even from about 0.2 wt % to upper levels of 0.5 or even 0.75 wt %. Soil release polymers—The detergent compositions of the present invention may also include one or more soil release polymers which aid the removal of soils from fabrics such as cotton and polyester based fabrics, in particular the removal of hydrophobic soils from polyester based fabrics. The soil release polymers may for example be nonionic or anionic terephthalte based polymers, polyvinyl caprolactam and related copolymers, vinyl graft copolymers, polyester polyamides see for example Chapter 7 in Powdered Detergents, Surfactant science series volume 71, Marcel Dekker, Inc. Another type of soil release polymers are amphiphilic alkoxylated grease cleaning polymers comprising a core structure and a plurality of alkoxylate groups attached to that core structure. The core structure may comprise a polyalkylenimine structure or a polyalkanolamine structure as described in detail in WO 2009/087523 (hereby incorporated by reference). Furthermore random graft co-polymers are suitable soil release polymers. Suitable graft co-polymers are described in more detail in WO 2007/138054, WO 2006/108856 and WO 2006/113314 (hereby incorporated by reference). Other soil release polymers are substituted polysaccharide structures especially substituted cellulosic structures such as modified cellulose deriviatives such as those described in EP 1867808 or WO 2003/040279 (both are hereby incorporated by reference). Suitable cellulosic polymers include cellulose, cellulose ethers, cellulose esters, cellulose amides and mixtures thereof. Suitable cellulosic polymers include anionically modified cellulose, nonionically modified cellulose, cationically modified cellulose, zwitterionically modified cellulose, and mixtures thereof. Suitable cellulosic polymers include methyl cellulose, carboxy methyl cellulose, ethyl cellulose, hydroxyl ethyl cellulose, hydroxyl propyl methyl cellulose, ester carboxy methyl cellulose, and mixtures thereof.

Anti-redeposition agents—The detergent compositions of the present invention may also include one or more anti-redeposition agents such as carboxymethylcellulose (CMC), polyvinyl alcohol (PVA), polyvinylpyrrolidone (PVP), polyoxyethylene and/or polyethyleneglycol (PEG), homopolymers of acrylic acid, copolymers of acrylic acid and maleic acid, and ethoxylated polyethyleneimines. The cellulose based polymers described under soil release polymers above may also function as anti-redeposition agents.

Other suitable adjunct materials include, but are not limited to, anti-shrink agents, anti-wrinkling agents, bactericides, binders, carriers, dyes, enzyme stabilizers, fabric softeners, fillers, foam regulators, hydrotropes, perfumes, pigments, sod suppressors, solvents, and structurants for liquid detergents and/or structure elasticizing agents.

### Formulation of Detergent Products

The detergent composition of the invention may be in any convenient form, e.g., a bar, a homogenous tablet, a tablet having two or more layers, a pouch having one or more compartments, a regular or compact powder, a granule, a paste, a gel, or a regular, compact or concentrated liquid.

Detergent formulation forms: Layers (same or different phases), Pouches, versus forms for Machine dosing unit.

Pouches can be configured as single or multicompartments. It can be of any form, shape and material which is suitable for hold the composition, e.g. without allowing the release of the composition to release of the composition from the pouch prior to water contact. The pouch is made from water soluble film which encloses an inner volume. Said inner volume can be divided into compartments of the pouch. Preferred films are polymeric materials preferably polymers which are formed into a film or sheet. Preferred polymers, copolymers or derivates thereof are selected polyacrylates, and water soluble acrylate copolymers, methyl cellulose, carboxy methyl cellulose, sodium dextrin, ethyl cellulose, hydroxyethyl cellulose, hydroxypropyl methyl cellulose, malto dextrin, poly methacrylates, most preferably polyvinyl alcohol copolymers and, hydroxypropyl methyl cellulose (HPMC). Preferably the level of polymer in the film for example PVA is at least about 60%. Preferred average molecular weight will typically be about 20,000 to about 150,000. Films can also be of blended compositions comprising hydrolytically degradable and water soluble polymer blends such as polylactide and polyvinyl alcohol (known under the Trade reference M8630 as sold by MonoSol LLC, Indiana, USA) plus plasticisers like glycerol, ethylene glycerol, propylene glycol, sorbitol and mixtures thereof. The pouches can comprise a solid laundry cleaning composition or part components and/or a liquid cleaning composition or part components separated by the water soluble film. The compartment for liquid components can be different in composition than compartments containing solids. Ref: (US2009/0011970 A1).

Detergent ingredients can be separated physically from each other by compartments in water dissolvable pouches or in different layers of tablets. Thereby negative storage interaction between components can be avoided. Different dissolution profiles of each of the compartments can also give rise to delayed dissolution of selected components in the wash solution.

### DEFINITION/CHARACTERISTICS OF THE FORMS

A liquid or gel detergent, which is not unit dosed, may be aqueous, typically containing at least 20% by weight and up to 95% water, such as up to about 70% water, up to about 65% water, up to about 55% water, up to about 45% water, up to about 35% water. Other types of liquids, including without limitation, alkanols, amines, diols, ethers and polyols may be included in an aqueous liquid or gel. An aqueous liquid or gel detergent may contain from 0-30% organic solvent.

A liquid or gel detergent may be non-aqueous.

### Laundry Soap Bars

The enzymes of the invention may be added to laundry soap bars and used for hand washing laundry, fabrics and/or textiles. The term laundry soap bar includes laundry bars, soap bars, combo bars, syndet bars and detergent bars. The types of bar usually differ in the type of surfactant they contain, and the term laundry soap bar includes those containing soaps from fatty acids and/or synthetic soaps. The laundry soap bar has a physical form which is solid and not a liquid, gel or a powder at room temperature. The term solid is defined as a physical form which does not significantly change over time, i.e. if a solid object (e.g. laundry soap bar) is placed inside a container, the solid object does not change to fill the container it is placed in. The bar is a solid typically in bar form but can be in other solid shapes such as round or oval.

The laundry soap bar may contain one or more additional enzymes, protease inhibitors such as peptide aldehydes (or hydrosulfite adduct or hemiacetal adduct), boric acid, borate, borax and/or phenylboronic acid derivatives such as 4-formylphenylboronic acid, one or more soaps or synthetic surfactants, polyols such as glycerine, pH controlling compounds such as fatty acids, citric acid, acetic acid and/or formic acid, and/or a salt of a monovalent cation and an organic anion wherein the monovalent cation may be for example Na⁺, K⁺ or NH₄⁺ and the organic anion may be for example formate, acetate, citrate or lactate such that the salt of a monovalent cation and an organic anion may be, for example, sodium formate.

The laundry soap bar may also contain complexing agents like EDTA and HEDP, perfumes and/or different type of fillers, surfactants e.g. anionic synthetic surfactants, builders, polymeric soil release agents, detergent chelators, stabilizing agents, fillers, dyes, colorants, dye transfer inhibitors, alkoxylated polycarbonates, suds suppressers, structurants, binders, leaching agents, bleaching activators, clay soil removal agents, anti-redeposition agents, polymeric dispersing agents, brighteners, fabric softeners, perfumes and/or other compounds known in the art.

The laundry soap bar may be processed in conventional laundry soap bar making equipment such as but not limited to: mixers, plodders, e.g a two stage vacuum plodder, extruders, cutters, logo-stampers, cooling tunnels and wrappers. The invention is not limited to preparing the laundry soap bars by any single method. The premix of the invention may be added to the soap at different stages of the process. For example, the premix containing a soap, an enzyme, optionally one or more additional enzymes, a protease inhibitor, and a salt of a monovalent cation and an organic anion may be prepared and and the mixture is then plodded. The enzyme and optional additional enzymes may be added at the same time as the protease inhibitor for example in liquid form. Besides the mixing step and the plodding step, the process may further comprise the steps of milling, extruding, cutting, stamping, cooling and/or wrapping.

### Granular Detergent Formulations

A granular detergent may be formulated as described in WO09/092699, EP1705241, EP1382668, WO07/001262, U.S. Pat. No. 6,472,364, WO04/074419 or WO09/102854. Other useful detergent formulations are described in WO09/124162, WO09/124163, WO09/117340, WO09/117341, WO09/117342, WO09/072069, WO09/063355, WO09/132870, WO09/121757, WO09/112296, WO09/112298, WO09/103822, WO09/087033, WO09/050026, WO09/047125, WO09/047126, WO09/047127, WO09/047128, WO09/021784, WO09/010375, WO09/000605, WO09/122125, WO09/095645, WO09/040544, WO09/040545, WO09/024780, WO09/004295, WO09/004294, WO09/121725, WO09/115391, WO09/115392, WO09/074398, WO09/074403, WO09/068501, WO09/065770, WO09/021813, WO09/030632, and WO09/015951.

WO2011025615, WO2011016958, WO2011005803, WO2011005623, WO2011005730, WO2011005844, WO2011005904, WO2011005630, WO2011005830, WO2011005912, WO2011005905, WO2011005910, WO2011005813, WO2010135238, WO2010120863, WO2010108002, WO2010111365, WO2010108000, WO2010107635, WO2010090915, WO2010033976, WO2010033746, WO2010033747, WO2010033897, WO2010033979, WO2010030540, WO2010030541, WO2010030539, WO2010024467, WO2010024469, WO2010024470, WO2010025161, WO2010014395, WO2010044905,

WO2010145887, WO2010142503, WO2010122051, WO2010102861, WO2010099997, WO2010084039, WO2010076292, WO2010069742, WO2010069718, WO2010069957, WO2010057784, WO2010054986, WO2010018043, WO2010003783, WO2010003792,

WO2011023716, WO2010142539, WO2010118959, WO2010115813, WO2010105942, WO2010105961, WO2010105962, WO2010094356, WO2010084203, WO2010078979, WO2010072456, WO2010069905, WO2010076165, WO2010072603, WO2010066486, WO2010066631, WO2010066632, WO2010063689, WO2010060821, WO2010049187, WO2010031607, WO2010000636,

### Uses

The present invention is directed to methods for using the polypeptides having alpha-amylase activity, or compositions thereof, in a cleaning process such as laundry or hard surface cleaning including automated dish wash.

The soils and stains that are important for cleaning are composed of many different substances, and a range of different enzymes, all with different substrate specificities, have been developed for use in detergents both in relation to laundry and hard surface cleaning, such as dishwashing. These enzymes are considered to provide an enzyme detergency benefit, since they specifically improve stain removal in the cleaning process that they are used in, compared to the same process without enzymes. Stain removing enzymes that are known in the art include enzymes such as proteases, amylases, lipases, cutinases, cellulases, endoglucanases, xyloglucanases, pectinases, pectin lyases, xanthanases, peroxidaes, haloperoxygenases, catalases and mannanases.

In one aspect, the invention concerns the use of alpha-amylases of the present invention in detergent compositions, for use in cleaning hard-surfaces, such as dish wash, or in laundering or for stain removal. In an additional aspect, the present invention demonstrates that the use of the alpha amylases of the invention have an improved wash performance in detergent compositions and in detergent applications, such as dish wash or laundering at low temperatures.

In a further aspect, the present invention demonstrates that the use of alpha-amylases of the invention have an improved wash performance in detergent compositions at low temperature washing, such as at 15 degrees C.

Another aspect of the invention is the use of the detergent composition comprising an alpha-amylase of the present invention together with one or more surfactants and optionally one or more detergent components, selected from the list comprising of hydrotropes, builders and co-builders, bleaching systems, polymers, fabric hueing agents and adjunct materials, or any mixture thereof in detergent compositions and in detergent applications.

A further aspect is the use of the detergent composition comprising an alpha-amylase of the present invention together with one or more surfactants, and one or more additional enzymes selected from the group comprising of proteases, lipases, cutinases, cellulases, endoglucanases, xyloglucanases, pectinases, pectin lyases, xanthanases, peroxidaes, haloperoxygenases, catalases and mannanases, or any mixture thereof in detergent compositions and in detergent applications.

In another aspect, the invention relates to a laundering process which can be for household laundering as well as industrial laundering. Furthermore, the invention relates to a process for the laundering of textiles (e.g. fabrics, garments, cloths etc.) where the process comprises treating the textile with a washing solution containing a detergent composition and an alpha-amylase of the present invention. The laundering can for example be carried out using a household or an industrial washing machine or be carried out by hand using a detergent composition containing a glucoamylase of the invention.

In another aspect, the invention relates to a dish wash process which can be for household dish wash as well as industrial dish wash. Furthermore, the invention relates to a process for the washing of hard surfaces (e.g. cutlery such as knives, forks, spoons; crockery such as plates, glasses, bowls; and pans) where the process comprises treating the hard surface with a washing solution containing a detergent composition and an alpha-amylases of the present invention. The hard surface washing can for example be carried out using a household or an industrial dishwasher or be carried out by hand using a detergent composition containing an alpha-amylase of the invention, optionally together with one or more further enzymes selected from the group comprising of proteases, amylases, lipases, cutinases, cellulases, endoglucanases, xyloglucanases, pectinases, pectin lyases, xanthanases, peroxidaes, haloperoxygenases, catalases, mannanases, or any mixture thereof.

In a further aspect, the invention relates to a method for removing a stain from a surface comprising contacting the surface with a composition comprising an alpha-amylase of the present invention together with one or more surfactants and optionally one or more detergent components, selected from the list comprising of hydrotropes, builders and co-builders, bleaching systems, polymers, fabric hueing agents and adjunct materials, or any mixture thereof in detergent compositions and in detergent applications. A further aspect is a method for removing a stain from a surface comprising contacting the surface with a composition comprising an alpha-amylase of the present invention together with one or more surfactants, one or more additional enzymes selected from the group comprising of proteases, lipases, cutinases, cellulases, endoglucanases, xyloglucanases, pectinases, pectin lyases, xanthanases, peroxidaes, haloperoxygenases, catalases and mannanases, or any mixture thereof in detergent compositions and in detergent applications.

The polypeptides of the present invention may thus be added to and become a component of a detergent composition.

The detergent composition of the present invention may be formulated, for example, as a hand or machine laundry detergent composition including a laundry additive composition suitable for pre-treatment of stained fabrics and a rinse added fabric softener composition, or be formulated as a detergent composition for use in general household hard surface cleaning operations, or be formulated for hand or machine dishwashing operations.

In a specific aspect, the present invention provides a detergent additive comprising a polypeptide of the present invention as described herein.

Examples are given below of preferred uses of the compositions of the present invention. The dosage of the composition and other conditions under which the composition is used may be determined on the basis of methods known in the art.

### EXAMPLES

### Strains

Tenacibaculum geojense, isolated from a public beach in the USA.

Tenacibaculum sp-62066 and Ahrensia sp-62069 are isolated from mud samples from Denmark.

### Media and Solutions

LB plates containing 6 μg/l chloramphenicol and AZCL amylose (Megazyme, Wicklow, Ireland).

### Method for Measuring Alpha-Amylase

pNP-G7 Assay for Determination of Alpha-Amylase Activity

The alpha-amylase activity may be determined by a method employing the G7-pNP substrate. G7-pNP which is an abbreviation for 4,6-ethylidene(G₇)-p-nitrophenyl(G₁)-α,D-maltoheptaoside, a blocked oligosaccharide which can be cleaved by an endo-amylase, such as an alpha-amylase. Following the cleavage, the alpha-Glucosidase included in the kit digest the hydrolysed substrate further to liberate a free PNP molecule which has a yellow color and thus can be measured by visible spectophometry at λ=405 nm (400-420 nm.). Kits containing G7-pNP substrate and alpha-Glucosidase is manufactured by Roche/Hitachi (cat. No. 11876473).

### Reagents:

The G7-pNP substrate from this kit contains 22 mM 4,6-ethylidene-G7-pNP and 52.4 mM HEPES (2-[4-(2-hydroxyethyl)-1-piperazinyl]-ethanesulfonic acid), pH 7.0).

The alpha-Glucosidase reagent contains 52.4 mM HEPES, 87 mM NaCl, 12.6 mM MgCl₂, 0.075 mM CaCl₂, ≧4 kU/L alpha-glucosidase).

The substrate working solution is made by mixing 1 mL of the alpha-Glucosidase reagent with 0.2 mL of the G7-pNP substrate. This substrate working solution is made immediately before use.

Dilution buffer: 50 mM MOPS, 0.05% (w/v) Triton X100 (polyethylene glycol p-(1,1,3,3-tetramethylbutyl)-phenyl ether (C₁₄H₂₂O(C₂H₄O)ₙ (n=9-10))), 1 mM CaCl2, pH8.0.

### Procedure:

The amylase sample to be analyzed was diluted in dilution buffer to ensure the pH in the diluted sample is 7. The assay was performed by transferring 20 μl diluted enzyme samples to 96 well microtiter plate and adding 80 μl substrate working solution. The solution was mixed and pre-incubated 1 minute at room temperature and absorption is measured every 20 sec. over 5 minutes at OD 405 nm.

The slope (absorbance per minute) of the time dependent absorption-curve is directly proportional to the specific activity (activity per mg enzyme) of the alpha-amylase in question under the given set of conditions. The amylase sample should be diluted to a level where the slope is below 0.4 absorbance units per minute.

### Wash Performance of Alpha-Amylases Using Automatic Mechanical Stress Assay

In order to assess the wash performance of the alpha-amylases in a detergent base composition, washing experiments may be performed using Automatic Mechanical Stress Assay (AMSA). With the AMSA test the wash performance of a large quantity of small volume enzyme-detergent solutions can be examined. The AMSA plate has a number of slots for test solutions and a lid firmly squeezing the textile swatch to be washed against all the slot openings. During the washing time, the plate, test solutions, textile and lid are vigorously shaken to bring the test solution in contact with the textile and apply mechanical stress in a regular, periodic oscillating manner. For further description see WO 02/42740, especially the paragraph “Special method embodiments” at page 23-24.

### General Wash Performance Description

A test solution comprising water (6° dH), 0.79 g/L detergent, e.g. model detergent J as described below, and the enzyme of the invention at concentration of 0, 0.3 or 0.6 mg enzyme protein/L, is prepared. Fabrics stained with starch (CS-28 from Center For Test materials BV, P.O. Box 120, 3133 KT, Vlaardingen, The Netherlands) is added and washed for 20 minutes at 15° C. or 30° C. After thorough rinse under running tap water and drying in the dark, the light intensity values of the stained fabrics are subsequently measured as a measure for wash performance. The test with 0 mg enzyme protein/L is used as a blank and corresponds to the contribution from the detergent. Preferably mechanical action is applied during the wash step, e.g. in the form of shaking, rotating or stirring the wash solution with the fabrics. The AMSA wash performance experiments were conducted under the experimental conditions specified below:

TABLE A
Experimental condition

| | |
|---|---|
| Detergent | Model detergent J (see Table B) |
| Detergent dosage | 0.79 g/L |
| Test solution volume | 160 micro L |
| pH | As is |
| Wash time | 20 minutes |
| Temperature | 15° C. or 30° C. |
| Water hardness | 6° dH |
| Enzyme concentration in test | 0.3 mg/L or 0.6 mg/L |
| Test material | CS-28 (Rice starch cotton) |

TABLE B
Model detergent J

| | Content of compound | % active component |
|---|---|---|
| Compound | (% w/w) | (% w/w) |
| LAS | 5.15 | 5.00 |
| AS | 5.00 | 4.50 |
| AEOS | 14.18 | 10.00 |
| Coco fatty acid | 1.00 | 1.00 |
| AEO | 5.00 | 5.00 |
| MEA | 0.30 | 0.30 |
| MPG | 3.00 | 3.00 |
| Ethanol | 1.50 | 1.35 |
| DTPA (as Na5 salt) | 0.25 | 0.10 |
| Sodium citrate | 4.00 | 4.00 |
| Sodium formate | 1.00 | 1.00 |
| Sodium hydroxide | 0.66 | 0.66 |
| H₂O, ion exchanged | 58.95 | 58.95 |

Water hardness was adjusted to 6° dH by addition of CaCl₂, MgCl₂, and NaHCO₃ (Ca²⁺:Mg²⁺:HCO₃_{^{−}}=2:1:4.5) to the test system. After washing the textiles were flushed in tap water and dried.

TABLE C
Experimental condition

| | |
|---|---|
| Detergent | Model detergent A (see Table D) |
| Detergent dosage | 3.33 g/L |
| Test solution volume | 160 micro L |
| pH | As is |
| Wash time | 20 minutes |
| Temperature | 15° C. or 30° C. |
| Water hardness | 15° dH |
| Enzyme concentration in test | 0.3 mg/L or 0.6 mg/L |
| Test material | CS-28 (Rice starch cotton) |

TABLE D
Model detergent A

| | Content of compound | % active component |
|---|---|---|
| Compound | (% w/w) | (% w/w) |
| LAS | 12.00 | 11.60 |
| AEOS, SLES | 17.63 | 4.90 |
| Soy fatty acid | 2.75 | 2.48 |
| Coco fatty acid | 2.75 | 2.80 |
| AEO | 11.00 | 11.00 |
| Sodium hydroxide | 1.75 | 1.80 |
| Ethanol/Propan-2-ol | 3.00 | 2.70/0.30 |
| MPG | 6.00 | 6.00 |
| Glycerol | 1.71 | 1.70 |
| TEA | 3.33 | 3.30 |
| Sodium formate | 1.00 | 1.00 |
| Sodium citrate | 2.00 | 2.00 |
| DTMPA | 0.48 | 0.20 |
| PCA | 0.46 | 0.18 |
| Phenoxy ethanol | 0.50 | 0.50 |
| H₂O, ion exchanged | 33.64 | 33.64 |

Water hardness was adjusted to 15° dH by addition of CaCl₂, MgCl₂, and NaHCO₃ (Ca²⁺:Mg²⁺:HCO₃_{^{−}}=4:1:7.5) to the test system. After washing the textiles were flushed in tap water and dried.

TABLE E
Experimental condition

| | |
|---|---|
| Detergent | Model detergent X (see Table F) |
| Detergent dosage | 1.75 g/L |
| Test solution volume | 160 micro L |
| pH | As is |
| Wash time | 20 minutes |
| Temperature | 15° C. or 30° C. |
| Water hardness | 12° dH |
| Enzyme concentration in test | 0.3 mg/L or 0.6 mg/L |
| Test material | CS-28 (Rice starch cotton) |

TABLE F
Model detergent X

| | Content of compound | % active component |
|---|---|---|
| Compound | (% w/w) | (% w/w) |
| LAS | 16.50 | 15.00 |
| AEO* | 2.00 | 2.00 |
| Sodium carbonate | 20.00 | 20.00 |
| Sodium (di)silicate | 12.00 | 9.90 |
| Zeolite A | 15.00 | 12.00 |
| Sodium sulfate | 33.50 | 33.50 |
| PCA | 1.00 | 1.00 |

*Model detergent X is mixed without AEO. AEO is added separately before wash.

Water hardness was adjusted to 12° dH by addition of CaCl₂, MgCl₂, and NaHCO₃ (Ca²⁺:Mg²⁺:HCO₃_{^{−}}=2:1:4.5) to the test system. After washing the textiles were flushed in tap water and dried.

TABLE G
Experimental condition

| | |
|---|---|
| Detergent | Model detergent T (see Table H) |
| Detergent dosage | 5.33 g/L |
| Test solution volume | 160 micro L |
| pH | As is |
| Wash time | 20 minutes |
| Temperature | 15° C. or 30° C. |
| Water hardness | 15° dH |
| Enzyme concentration in test | 0.3 mg/L or 0.6 mg/L |
| Test material | CS-28 (Rice starch cotton) |

TABLE H
Model detergent T

| | Content of compound | % active component |
|---|---|---|
| Compound | (% w/w) | (% w/w) |
| LAS, sodium salt | 11.00 | 10.00 |
| AS/AEOS, sodium salt | 2.00 | 1.80 |
| Soap, sodium salt | 2.00 | 2.00 |
| AEO* | 3.00 | 3.00 |
| Sodium carbonate | 15.15 | 14.90 |
| Sodium silicate | 3.00 | 2.50 |
| Zeolite A | 18.75 | 15.00 |
| HEDP-Na₄ | 0.15 | 0.13 |
| Sodium citrate | 2.00 | 2.00 |
| AA/MA copolymer, | 1.65 | 1.50 |
| sodium salt | | |
| CMC | 2.50 | 1.60 |
| SRP | 0.50 | 0.50 |
| Sodium sulfate | 36.30 | 35.80 |
| Silicone | 2.00 | 2.00 |

*Model detergent T is mixed without AEO. AEO is added separately before wash.

Water hardness was adjusted to 15° dH by addition of CaCl₂, MgCl₂, and NaHCO₃ (Ca²⁺:Mg²⁺:HCO₃_{^{−}}=4:1:7.5) to the test system. After washing the textiles were flushed in tap water and dried.

The wash performance is measured as the brightness expressed as the intensity of the light reflected from the sample when illuminated with white light. When the sample is stained the intensity of the reflected light is lower, than that of a clean sample. Therefore the intensity of the reflected light can be used to measure wash performance.

Color measurements are made with a professional flatbed scanner (Kodak iQsmart, Kodak) used to capture an image of the washed textile. To extract a value for the light intensity from the scanned images, 24-bit pixel values from the image are converted into values for red, green and blue (RGB). The intensity value (Int) is calculated by adding the RGB values together as vectors and then taking the length of the resulting vector:

Int=\sqrt{r²+g²+b²}

### Textile:

Textile sample CS-28 (rice starch on cotton) is obtained from Center For Testmaterials BV, P.O. Box 120, 3133 KT Vlaardingen, the Netherlands.

### Example 1: Cloning and Preparation of the Alpha-Amylase of SEQ ID NO: 2—Tenacibaculum geojense

### Identification of Alpha-Amylase Coding Gene

For the cloning of the alpha-amylase gene, the genomic DNA of Tenacibaculum geojense was sequenced. Chromosomal DNA of Tenacibaculum geojense was isolated by QIAamp DNA Blood Mini Kit” (Qiagen, Hilden, Germany). Five ug of chromosomal DNA was sent for genome sequencing at FASTERIS SA, Switzerland.

The alpha-amylase gene was discovered by homology searches in public protein databases, a technique that is known by the person skilled in the art. The predicted coding sequence is shown in SEQ ID NO: 1, and the encoded alpha-amylase in SEQ ID NO: 2. The alpha-amylase was found to have 79.5% sequence identity to the closest public protein sequence from Aquimarine agarilytica. The enzyme properties of Aquimarine agarilytica alpha-amylase are not known to-date, enzyme properties of Tenacibaculum geojense are disclosed in this application.

### Cloning and Expression of Alpha-Amylase Gene

The signal peptide from the alkaline alpha-amylase from B. licheniformis (amyL) was fused by SOE PCR fusion as described in WO 99/43835 (hereby incorporated by reference) in frame to the DNA encoding the alpha-amylase and replacing the genes native secretion signal. To amplify the coding DNA, genomic DNA of Tenacibaculum geojense was used as template and the oligomers ForwardPrimer and ReversePrimer to amplify the gene by PCR.

Forward Primer:
(SEQ ID NO: 3)
TGCCTCATTC TGCAGCCGCG CAAGACGAAG ATGTACTATT TCA
Reverse Primer:
(SEQ ID NO: 4)
TCATTAGTGG TGATGGTGAT GATGTTGTGT CCAAACAGCA TAAT

The derived PCR product was fused to expression cassette elements. The alpha-amylase gene from Tenacibaculum geojense was expressed by control of a triple promoter system consisting of the promoters from Bacillus licheniformis alpha-amylase gene (amyL), Bacillus amyloliquefaciens alpha-amylase gene (amyQ), and the Bacillus thuringiensis cryIIIA promoter including stabilizing sequence. The expression cassette has been described in WO 99/43835. Furthermore, the expression cassette contained a terminator (term) sequence and a gene coding for chloramphenicol acetyltransferase (cam) which was used as selection maker (as described in (Diderichsen et al., 1993, Plasmid 30: 312-315) for B. subtilis.

The complete expression cassette described above was transformed into B. subtilis and the expression cassette was integrated into the Bacillus subtilis chromosome by homologous recombination into the pectate lyase gene locus (WO 99/43835).

Chloramphenicol resistant transformants were analyzed by DNA sequencing to verify the correct DNA sequence of the construct. The translated protein sequence corresponds to SEQ ID NO: 2.

Transformants were plated on LB plates containing 6 μg/l chloramphenicol and cibacron dyed amylopectin in duplicate. The plates were incubated over night at 37° C. After the incubation clearing zones appeared around transformants expressing active alpha-amylase. Two alpha-amylase expressing clones were selected for further characterization.

One clone with confirmed gene sequence was incubated into a deep well microtiter plate containing 2 ml liquid medium and was shaken at 300 rpm at 26° C. At day 3 the cultures were harvested by centrifugation, 200 μl of the supernatants were collected for SDS-gel electrophoresis.

The samples for SDS gel electrophoresis were mixed with 180 μl Novex Tricine SDS sample buffer 2× (Invitrogen, Cat. no. LC1676) and 20 μl NuPage sample reducing agent (Invitrogen, Cat No. NP0009), and 10 μl of each were loaded on a 8-16% Stainfree Tris-HCl precase Criterion gel (Biorad) and run in 1× Tris/Glycine SDS buffer according to the manufacturers instructions. The gel was visualized using an Imager together with BlueStain for gel staining. The results showed a clear recombinant protein band in the expected size. The enzyme was purified as described in Example 3.

### Example 2: Cloning and Preparation of the Alpha-Amylase of SEQ ID NO: 9 and 14—Alpha-Amylase of SEQ ID NO: 9—Tenacibaculum sp-62066

### Identification of Alpha-Amylase Coding Gene

For the cloning of the alpha-amylase gene, the genomic DNA of Tenacibaculum sp-62066 was sequenced. Chromosomal DNA of Tenacibaculum sp-62066 was isolated by QIAamp DNA Blood Mini Kit” (Qiagen, Hilden, Germany). Five ug of chromosomal DNA was sent for genome sequencing at FASTERIS SA, Switzerland.

The alpha-amylase gene was discovered by homology searches in public protein databases, a technique that is known by the person skilled in the art. The coding sequence is shown in SEQ ID NO: 5 and the alpha-amylase in SEQ ID NO: 6. The alpha-amylase was found to have 80% sequence identity to the closest public protein sequence from Aquimarina agarilytica.

### Cloning and Expression of Alpha-Amylase Gene

Based on the nucleotide sequence identified as SEQ ID NO: 5, a codon optimized synthetic gene having SEQ ID NO: 7, was synthesized by Gene Art (GENEART AG BioPark, Josef-Engert-Str. 11, 93053, Regensburg, Germany). The synthetic gene was subcloned using Clal and Mlul restriction sites into a Bacillus expression vector as described in WO 12/025577. The alpha-amylase was expressed with a Bacillus clausii secretion signal (with the following amino acid sequence: MKKPLGKIVASTALLISVAFSSSIASA, nucleotides 1-81 of SEQ ID NO: 7) replacing the native secretion signal. The amylase was expressed with a C-terminal His-tag (HHHHHH) to ease purification. The expressed DNA sequence is listed in SEQ ID NO:7 and the encoded protein in SEQ ID NO:8 and the expressed mature protein sequence in SEQ ID NO: 9. The expression plasmid was transformed into Bacillus subtilis. The expression cassette was integrated by homologous recombination into the pectate lyase locus. Transformants were selected on LB plates supplemented with 6 μg/ml chloramphenicol. The recombinant Bacillus subtilis clone containing the integrated expression construct was selected and cultivated on a rotary shaking table in 500 mL baffled Erlenmeyer flasks each containing 100 ml yeast extract-based media. The clone was cultivated for 3 days at 26° C. The enzyme containing supernatants were harvested and the enzyme purified as described in Example 4.

Alpha-Amylase of SEQ ID NO: 14—Ahrensia sp-62069

### Identification of Alpha-Amylase Coding Gene

For the cloning of the alpha-amylase gene, the genomic DNA of Ahrensia sp-62069 was sequenced. Chromosomal DNA of Ahrensia sp-62069 was isolated by QIAamp DNA Blood Mini Kit” (Qiagen, Hilden, Germany). Five ug of chromosomal DNA was sent for genome sequencing at FASTERIS SA, Switzerland.

The alpha-amylase gene was discovered by homology searches in public protein databases, a technique that is known by the person skilled in the art. The coding sequence is shown in SEQ ID NO: 10. The alpha-amylase was found to have 79.7% sequence identity to the closest public protein sequence from Aquimarina agarilytica.

### Cloning and Expression of Alpha-Amylase Gene

Based on the nucleotide sequence identified as SEQ ID NO: 10, a codon optimized synthetic gene having SEQ ID NO: 12, was synthesized by Gene Art (GENEART AG BioPark, Josef-Engert-Str. 11, 93053, Regensburg, Germany). The synthetic gene was subcloned using Clal and Mlul restriction sites into a Bacillus expression vector as described in WO 12/025577. The alpha-amylase was expressed with a Bacillus clausii secretion signal (with the following amino acid sequence: MKKPLGKIVASTALLISVAFSSSIASA) replacing the native secretion signal. The amylase was expressed with a C-terminal His-tag (HHHHHH) to ease purification. The expressed DNA sequence is listed in SEQ ID NO:12 and the encoded protein in SEQ ID NO:13 and the expressed mature protein sequence in SEQ ID NO:14. The expression plasmid was transformed into Bacillus subtilis. The expression cassette was integrated by homologous recombination into the pectate lyase locus. Transformants were selected on LB plates supplemented with 6 μg/ml chloramphenicol. The recombinant Bacillus subtilis clone containing the integrated expression construct was selected and cultivated on a rotary shaking table in 500 mL baffled Erlenmeyer flasks each containing 100 ml yeast extract-based media. The clone was cultivated for 3 days at 26° C. The enzyme containing supernatants were harvested and the enzyme purified as described in Example 4.

### Example 3: Purification of Alpha-Amylase of SEQ ID NO: 2

The isolated expression clone was selected and was cultivated on a rotary shaking table in 500 mL baffled Erlenmeyer flasks each containing 100 ml Cal18 medium. The clone was cultivated for 3 days at 26° C. whereafter the supernatants were collected for purification of alpha-amylase.

800 ml supernatant from the selected expression clone grown in shake flasks, prepared as described in example 1, was used for purification of the alpha-amylase. The supernatant was filtered through a 0.45 μl filer and subsequent through a EKS filter on a pressurized nut.

pH was adjusted to 8.0 and the supernatant was loaded on a 70 ml Chelating sepharose column loaded with CuSO₄. The column was eluated using a gradient of 0% to 100% over 5 column volumes of buffer A: 50 mM Tris-HCl, 0.1 mM CaCl₂, pH and buffer B: 50 mM MES, 0.5 M imidazole, 0.1 mM CaCl₂, pH 7.0, with collection of fractions of 10 ml. The flow rate was 10 ml/min. Fractions 17-23 were pooled based on the chromatogram and the buffer was changed into 50 mM MES, 0.1 mM CaCl₂, pH 7.0 by dialysis. Subsequently the pool was loaded on a 30 ml Sp-Sepharose column, which was eluted using a gradient of 0 to 100% over 5 column volumes of Buffer A: 50 mM MES, 0.1 mM CaCl₂, pH 7.0 and buffer B: 50 mM MES, 0.1 mM CaCl₂, 1 M NaCl, pH 7.0, with collection of 10 ml fractions and a flow rate of 10 ml/min. Samples of each fraction was run on a SDS gel as described above and the fractions 7-9 with a clear band in the expected size were pooled and stored for further analysis of the enzyme.

Alpha-amylase activity of the purified enzymes was determined using the method described above and it was found that the purified enzyme was active.

### Example 4: Purification of Alpha-Amylases of SEQ ID NO's 9 and 14

pH of the supernatants was adjusted to pH 8, filtrated through a 0.2 μM filter, and the supernatant applied to a 5 ml HisTrap™ excel column. Prior to loading, the column had been equilibrated in 5 column volumes (CV) of 50 mM Tris/HCl pH 8. In order to remove unbound material, the column was washed with 8 CV of 50 mM Tris/HCl pH 8, and elution of the target was obtained with 50 mM HEPES pH 7+10 mM imidazole. The eluted protein was desalted on a HiPrep™ 26/10 desalting column, equilibrated using 3 CV of 50 mM HEPES pH 7+100 mM NaCl. This buffer was also used for elution of the target, and the flow rate was 10 ml/min. Relevant fractions were selected and pooled based on the chromatogram and SDS-PAGE analysis.

### Example 5: Wash Performance of the Mature Alpha-Amylase of SEQ ID NO: 2 (Amino Acids 24 to 428 of SEQ ID NO: 2) (0.3 mg EP/L)

The alpha-amylase of amino acids 24 to 428 of SEQ ID NO: 2 of the invention as prepared and purified in example 2 and 3 was tested in AMSA test as described above. The test was done using a test swatch CS-28 Rice starch on cotton (TEX353-5) provided by Center For Testmaterials BV, P.O. Box 120, 3133 KT Vlaardingen, the Netherlands. The enzyme was tested in four different detergent compositions: Model detergent A, Model detergent T, Model detergent J and Model detergent X and was done at both 15° C. and at 30° C.

The wash performance of the enzyme of the invention was compared with the performance of another wildtype alpha-amylase AA560 (SEQ ID NO: 15 herein), disclosed in WO 00/60060, that has been the basis for several detergent alpha-amylases, see e.g. WO 01/66712 and WO 06/02643.

The enzyme was dosed at 0.3 mg enzyme protein/I wash solution, and the results was calculated as an average of 4 determinations. Following results were obtained:

| | Intensity | | Intensity | | Intensity | | Intensity | |
|---|---|---|---|---|---|---|---|---|
| | in Model | | in Model | | in Model | | in Model | |
| | detergent J | | detergent A | | detergent X | | detergent T | |
| Enzyme | 15° C. | 30° C. | 15° C. | 30° C. | 15° C. | 30° C. | 15° C. | 30° C. |
| Blank | 329 | 334 | 327 | 336 | 329 | 333 | 328 | 334 |
| (without | | | | | | | | |
| enzyme) | | | | | | | | |
| AA560 (prior | 335 | 346 | 332 | 346 | 332 | 344 | 333 | 348 |
| art alpha- | | | | | | | | |
| amylase, | | | | | | | | |
| SEQ ID NO: | | | | | | | | |
| 15) | | | | | | | | |
| Mature alpha- | 343 | 348 | 340 | 349 | 343 | 350 | 341 | 348 |
| amylase of | | | | | | | | |
| SEQ ID NO: 2 | | | | | | | | |
| (amino acids | | | | | | | | |
| 24 to 428) | | | | | | | | |

The results shows that the enzyme of the invention provides for a significant wash performance in all the tested detergent composition and at both the tested temperatures. Next, the performance of the enzyme of the invention is equal to or better than the prior art wildtype enzyme AA560 in all the tested conditions.

Further, the enzyme of the invention has a considerable better wash performance at low temperature compared with the prior art wildtype AA560, which is reflected by the wash performance at 15° C.

Finally, the enzyme of the invention has a high performance at both 15° C. and at 30° C. This can be seen by observing the Δ intensities (the measured intensities—the intensities using the detergent alone) at 15° C. and at 30° C.:

| | Δ Intensity | | Δ Intensity | | Δ Intensity | | Δ Intensity | |
|---|---|---|---|---|---|---|---|---|
| | in Model | | in Model | | in Model | | in Model | |
| | detergent J | | detergent A | | detergent X | | detergent T | |
| Enzyme | 15° C. | 30° C. | 15° C. | 30° C. | 15° C. | 30° C. | 15° C. | 30° C. |
| AA560 (prior | 6 | 12 | 5 | 10 | 3 | 11 | 5 | 14 |
| art alpha- | | | | | | | | |
| amylase) | | | | | | | | |
| Mature alpha- | 14 | 14 | 13 | 13 | 14 | 17 | 13 | 14 |
| amylase of | | | | | | | | |
| SEQ ID NO: 2 | | | | | | | | |
| (amino acids | | | | | | | | |
| 24 to 428) | | | | | | | | |

Here it is clear that the enzyme of the invention has approximately same wash performance at 15° C. and at 30° C. in contrast to the prior art alpha-amylase AA560 where the performance at 15° C. is approximately half or less of the performance at 30° C.

| | 15/30 ratio | 15/30 ratio | 15/30 ratio | 15/30 ratio |
|---|---|---|---|---|
| | Model | Model | Model | Model |
| Enzyme | detergent J | detergent A | detergent X | detergent T |
| AA560 (prior | 0.50 | 0.50 | 0.27 | 0.36 |
| art alpha- | | | | |
| amylase) | | | | |
| Mature alpha- | 1.00 | 1.00 | 0.82 | 0.93 |
| amylase of | | | | |
| SEQ ID NO: 2 | | | | |
| (amino acids | | | | |
| 24 to 428) | | | | |

### Example 6: Wash Performance Compared to Commercial Detergent Alpha-Amylase Stainzyme™ (0.3 mg EP/L)

The wash performance was tested as described in Example 5 but compared to the commercial detergent alpha-amylase Stainzyme™

| | Intensity in | Intensity in | Intensity in | Intensity in |
|---|---|---|---|---|
| | Model | Model | Model | Model |
| | detergent J | detergent A | detergent X | detergent T |
| Enzyme | 15° C. | 15° C. | 15° C. | 15° C. |
| Blank | 329 | 327 | 329 | 328 |
| (without | | | | |
| enzyme) | | | | |
| Commercial | 337 | 336 | 340 | 339 |
| Alpha- | | | | |
| amylase | | | | |
| variant of | | | | |
| AA560 | | | | |
| (Stainzyme) | | | | |
| Mature alpha- | 343 | 340 | 343 | 341 |
| amylase of | | | | |
| SEQ ID NO: 2 | | | | |
| (amino acids | | | | |
| 24 to 428) | | | | |

| | Δ | Δ Intensity | Δ | Δ |
|---|---|---|---|---|
| | Intensity in | in | Intensity in | Intensity in |
| | Model | Model | Model | Model |
| | detergent J | detergent A | detergent X | detergent T |
| Enzyme | 15° C. | 15° C. | 15° C. | 15° C. |
| Commercial | 8 | 9 | 11 | 11 |
| Alpha- | | | | |
| amylase | | | | |
| variant of | | | | |
| AA560 | | | | |
| (Stainzyme) | | | | |
| Mature alpha- | 14 | 13 | 14 | 13 |
| amylase of | | | | |
| SEQ ID NO: 2 | | | | |
| (amino acids | | | | |
| 24 to 428) | | | | |

The results shows that the enzyme of the invention (amino acids 24 to 428 of SEQ ID NO: 2) provides for a considerably improved wash performance at 15° C. in all the tested detergent composition compared to the commercial alpha-amylase Stainzyme.

At 30° C. the results showed that Stainzyme had a better wash performance than that of the mature alpha-amylase of SEQ ID NO: 2 (amino acids 24 to 428).

### Example 7: Wash Performance of the Amylases (0.3 mg EP/L) of the Invention in Model Detergent A

The wash performance of the alpha-amylases of SEQ ID NO: 2 (amino acids 24 to 428), SEQ ID NO: 9 and SEQ ID NO: 14 as prepared and purified in example 2, 3 and 4 was tested in AMSA test as described above. The test was done using a test swatch CS-28 Rice starch on cotton (TEX353-5) provided by Center For Testmaterials BV, P.O. Box 120, 3133 KT Vlaardingen, the Netherlands. The enzyme was tested in Model detergent A at both 15° C. and at 30° C.

The wash performance of the enzymes of the invention was compared with the performance of another wildtype alpha-amylase AA560 (SEQ ID NO: 15 herein), disclosed in WO 00/60060, that has been the basis for several detergent alpha-amylases, see e.g. WO 01/66712 and WO 06/02643.

The enzyme was dosed at 0.3 mg enzyme protein/I wash solution, and the results was calculated as an average of 4 determinations. Following results were obtained:

| | Intensity in Model detergent A | |
|---|---|---|
| Enzyme | 15° C. | 30° C. |
| Blank (without enzyme) | 330 | 333 |
| AA560 (prior art alpha- | 335 | 347 |
| amylase) of SEQ ID NO: 15 | | |
| Mature alpha-amylase of SEQ ID | 341 | 353 |
| NO: 2 (amino acids 24 to 428) | | |
| SEQ ID NO: 9 | 338 | 349 |
| SEQ ID NO: 14 | 335 | 341 |

| | Δ Intensity in Model detergent A | |
|---|---|---|
| Enzyme | 15° C. | 30° C. |
| AA560 (prior art alpha- | 5 | 14 |
| amylase) of SEQ ID NO: 15 | | |
| Mature alpha-amylase of SEQ ID | 11 | 20 |
| NO: 2 (amino acids 24 to 428) | | |
| SEQ ID NO: 9 | 8 | 16 |
| SEQ ID NO: 14 | 5 | 8 |

The results shows that the enzymes of SEQ ID NO: 2 and 9 provides for an improved wash performance at 15° C. and 30° C. in model detergent A compared to the prior art amylase of SEQ ID NO: 15. The alpha-amylase of SEQ ID NO: 14 is on par at 15° C. The 15/30 ratio of the amylase of the prior art is 0.36 and is 0.55, 0.5 and 0.63 for the three enzymes of the invention.

### Example 8: Wash Performance of the Amylases (0.6 mg EP/L) of the Invention

The wash performance of the alpha-amylases of SEQ ID NO: 2 (amino acids 24 to 428), SEQ ID NO: 9 and SEQ ID NO: 14 as prepared and purified in example 2, 3 and 4 was tested in AMSA test as described above. The test was done using a test swatch CS-28 Rice starch on cotton (TEX353-5) provided by Center For Testmaterials BV, P.O. Box 120, 3133 KT Vlaardingen, the Netherlands. The enzyme was tested in four different detergent compositions: Model detergent A, Model detergent T, Model detergent J and Model detergent X and was done at both 15° C. and at 30° C.

The wash performance of the enzyme of the invention was compared with the performance of another wildtype alpha-amylase AA560 (SEQ ID NO: 15 herein), disclosed in WO 00/60060, that has been the basis for several detergent alpha-amylases, see e.g. WO 01/66712 and WO 06/02643.

The enzyme was dosed at 0.6 mg enzyme protein/L wash solution, and the results was calculated as an average of 4 determinations. Following results were obtained:

| | Intensity | | Intensity | | Intensity | | Intensity | |
|---|---|---|---|---|---|---|---|---|
| | in Model | | in Model | | in Model | | in Model | |
| | detergent J | | detergent A | | detergent X | | detergent T | |
| Enzyme | 15° C. | 30° C. | 15° C. | 30° C. | 15° C. | 30° C. | 15° C. | 30° C. |
| Blank | 332 | 334 | 330 | 333 | 331 | 336 | 330 | 334 |
| (without | | | | | | | | |
| enzyme) | | | | | | | | |
| AA560 (prior | 335 | 344 | 335 | 347 | 334 | 348 | 333 | 347 |
| art alpha- | | | | | | | | |
| amylase) | | | | | | | | |
| Mature alpha- | 334 | 340 | 341 | 353 | 340 | 345 | 339 | 343 |
| amylase of | | | | | | | | |
| SEQ ID NO: 2 | | | | | | | | |
| (amino acids | | | | | | | | |
| 24 to 428) | | | | | | | | |
| SEQ ID NO: 9 | 332 | 337 | 338 | 349 | 334 | 337 | 334 | 337 |
| SEQ ID NO: 14 | 332 | 335 | 335 | 341 | 334 | 337 | 333 | 334 |
| | Δ Intensity | | Δ Intensity | | Δ Intensity | | Δ Intensity | |
| | in Model | | in Model | | in Model | | in Model | |
| | detergent J | | detergent A | | detergent X | | detergent T | |
| Enzyme | 15° C. | 30° C. | 15° C. | 30° C. | 15° C. | 30° C. | 15° C. | 30° C. |
| AA560 (prior | 3 | 10 | 5 | 14 | 3 | 12 | 3 | 13 |
| art alpha- | | | | | | | | |
| amylase) | | | | | | | | |
| Mature alpha- | 2 | 6 | 11 | 20 | 9 | 9 | 9 | 9 |
| amylase of | | | | | | | | |
| SEQ ID NO: 2 | | | | | | | | |
| (amino acids | | | | | | | | |
| 24 to 428) | | | | | | | | |
| SEQ ID NO: 9 | 0 | 3 | 8 | 16 | 3 | 1 | 4 | 3 |
| SEQ ID NO: 14 | 0 | 1 | 5 | 8 | 3 | 1 | 3 | 0 |

The results of this experiment show that the enzymes of the invention provides for a significantly improved wash performance at 15° C. in model detergents A, X and T. This can be seen by observing the Δ intensities (the measured intensities—the intensities using the detergent alone) at 15° C.

It is also clear that the 15/30 ratio of the enzymes of the invention is significantly higher than that of the prior art amylase. Accordingly, for the enzymes of SEQ ID NO: 2 (amino acids 24 to 428), SEQ ID NOs: 9 and 14 the 15/30 ratio in model detergent A is respectively 0.55, 0.5 and 0.63. whereas that of the prior art alpha-amylase AA560 is only 0.36.

1. A polypeptide having alpha-amylase activity, and having a 15/30 ratio of at least 0.50, preferably at least 0.6, preferably at least 0.7, preferably at least 0.8, preferably at least 0.9 preferably at least 1.0, when measured in Model detergent A.

2. A polypeptide having alpha-amylase activity, selected from the group consisting of:
(a) a polypeptide having at least 85% sequence identity to the mature polypeptide of SEQ ID NO: 2;
(b) a polypeptide encoded by a polynucleotide that hybridizes under medium-high stringency conditions with (i) the mature polypeptide coding sequence of SEQ ID NO: 1, or (ii) the full-length complement thereof;
(c) a polypeptide encoded by a polynucleotide having at least 85% sequence identity to the mature polypeptide coding sequence of SEQ ID NO: 1;
(d) a variant of the mature polypeptide of SEQ ID NO: 2 comprising a substitution, deletion, and/or insertion at one or more positions;
(e) a polypeptide having at least 85% sequence identity to the mature polypeptide of SEQ ID NO: 6;
(f) a polypeptide encoded by a polynucleotide that hybridizes under medium-high stringency conditions with (i) the mature polypeptide coding sequence of SEQ ID NO: 5, or (ii) the full-length complement thereof;
(g) a polypeptide encoded by a polynucleotide having at least 85% sequence identity to the mature polypeptide coding sequence of SEQ ID NO: 5;
(h) a variant of the mature polypeptide of SEQ ID NO: 6 comprising a substitution, deletion, and/or insertion at one or more positions;
(i) a polypeptide having at least 85% sequence identity to the mature polypeptide of SEQ ID NO: 11;
(j) a polypeptide encoded by a polynucleotide that hybridizes under medium-high stringency conditions with (i) the mature polypeptide coding sequence of SEQ ID NO: 10, or (ii) the full-length complement thereof;
(k) a polypeptide encoded by a polynucleotide having at least 85% sequence identity to the mature polypeptide coding sequence of SEQ ID NO: 10;
(l) a variant of the mature polypeptide of SEQ ID NO: 11 comprising a substitution, deletion, and/or insertion at one or more positions; and
(m) a fragment of the polypeptide of (a), (b), (c), (d), (e), (f), (g), (h), (i), (j), (k) or (l) that has alpha-amylase activity.

3. The polypeptide of claim 2 having a 15/30 ratio of at least 0.50, preferably at least 0.6, preferably at least 0.7, preferably at least 0.8, preferably at least 0.9 or preferably at least 1.0, when measured in Model detergent A.

4. The polypeptide of claim 1, having at least 90%, at least 91%, at least 92%, at least 93%, at least 94%, at least 95%, at least 96%, at least 97%, at least 98%, at least 99% or 100% sequence identity to any of the mature polypeptides of SEQ ID NO: 2, 6 or 11.

5. The polypeptide of claim 1, which is encoded by a polynucleotide that hybridizes under high stringency or very high stringency conditions with any of the mature polypeptide coding sequences of SEQ ID NOs: 1, 5 or 10, or the full-length complement thereof.

6. The polypeptide of claim 1, which is encoded by a polynucleotide having at at least 90%, at least 91%, at least 92%, at least 93%, at least 94%, at least 95%, at least 96%, at least 97%, at least 98%, at least 99% or 100% sequence identity to the mature polypeptide coding sequence of any of SEQ ID NOs: 1, 5 and 10.

7. The polypeptide of claim 1 which is isolated.

8. The polypeptide of claim 1, comprising or consisting of SEQ ID NOs: 2, 6 or 11 or the mature polypeptide of SEQ ID NOs: 2, 6 or 11.

9. The polypeptide of claim 8, wherein the mature polypeptide of SEQ ID NO: 2 is amino acids 24 to 428 of SEQ ID NO: 2, the mature polypeptide of SEQ ID NO: 6 is amino acids 1 to 409 and the mature polypeptide of SEQ ID NO: 11 is amino acids 1 to 409.

10. The polypeptide of claim 1, which is a variant of the mature polypeptide of SEQ ID NO: 2, 6 or 11 comprising a substitution, deletion, and/or insertion at one or more positions.

11. The polypeptide of claim 8 which is a variant of the mature polypeptide of SEQ ID NO: 2.

12. A composition comprising the polypeptide of claim 1.

13. The composition of claim 12, which is a detergent composition comprising at least one surfactant.

14. The composition of claim 12, further comprising at least one additional enzyme selected among: protease, lipase, cutinase, an amylase, carbohydrase, cellulase, pectinase, mannanase, arabinase, galactanase, xylanase, oxidase, e.g., a laccase, and/or peroxidase.

15. (canceled)

16. An isolated polynucleotide encoding the polypeptide of claim 1

17. A nucleic acid construct or expression vector comprising the polynucleotide of claim 16 operably linked to one or more control sequences that direct the production of the polypeptide in an expression host.

18. A recombinant host cell comprising the polynucleotide of claim 16 operably linked to one or more control sequences that direct the production of the polypeptide.

19. A method of producing the polypeptide of claim 1, comprising cultivating a cell, which in its wild-type form produces the polypeptide, under conditions conducive for production of the polypeptide.

20. The method of claim 19, further comprising recovering the polypeptide.

21. A method of producing a polypeptide having alpha-amylase activity, comprising cultivating the host cell of claim 18 under conditions conducive for production of the polypeptide.

22. The method of claim 21, further comprising recovering the polypeptide.

23. A whole broth formulation or cell culture composition comprising a polypeptide of claim 1.


SEQUENCE LISTING (15 sequences)

SEQ ID NO: 1 - DNA, 2253 bp, Tenacibaculum geojense
  CDS (1)..(1284)
atgatgaagcaatgcctaaaaaaattgctcttatctttattgtttatcaatgcaataaatataagtgcacaagacgaagatgtactatttcaagcgtttgattggaatgtgcaaaatcaaccaactggtcaaacttggtttaatgtagtaactcagaatagttccgaaatcagtaatgctggttttgacatgatttggcttccacccgtaagtgattctggagcaccacaaggctacttaccaagagaactttacaattttaacagtgcatacggtacagaaagtcaacttagaaatttaattaatacctatcacaacttagatgttaaaattataggagatattgttattaatcatcgtgtaggtactaacgatgctgtaacttttacaaacccatcttggccaacaacttttattaccgcagatgatgaaggtagaaactttgttaattatcctgtagaatttagtataaatggtgattattttcctggaacagccttgaaagctgatggatctaacggaacttatggaccagcaagagatttagaccactataacccagctgttcgtcaagaaattaaaaattggatgaacttcttaaaaaacgatcttggttttgatggctggagatatgattttgttcatggttatgatcctatattcaacaaagagtataatgatgctacaaatccgtactttgctgtaggtgaactattagaaagtagtagaattcaaacaaataattggataaattttactcaacaatcatcttcagcttttgattttaatactaaagttacactacaaaacgcattaagagacaacaatttatcatatttaagagatggtaatggaaaaccttctggtatgataggaattaatcctactaaatctgttacctttttagataatcacgatacaggagcagcgcaacaatgctgtggatctggttatgtttttccaagtgatgaaaacaatcttagaaaaggttatgcctacattttaacacatcctggaaacccaatggtattttggacgcattattttgatgccccaataggtgttaaaaatgccataaaagatttaattagtattcgaaaagatgctagagtttttgcaggttcaaccataaatatagcagaagccagaaacgatttgtatgcggcttacatagatggtagaaacggtactattgcaatgaaactaggccgtggaaactggtctcctaatggtaataattgggttttaagaacatcaggaaatgattatgctgtttggacacaagggggaaaccctccaccatcagaagaaattacatttcgttttaaaaaacctaatgattggaatgcaaatattaatgcttatttttttgattcagcgagtaatagtacaatatcaggaacttctgaatggccaggacaaactatgaccagaatttcaggaaccccttggtatgaatatactttaaacgttccagttgggacatcaactaataacataaggttgatttttaatgatggtaataatcaaacagatgatttaagaagaggttctgacggatggtatgatatgggagtatggtctaattcatgtccgtctaactgtacaaacactcctccatctaattcaacagtaacttttaactttttaagaacttcttcttggggtaatacagcaaatgtttatttatatgataaaaatacaaacactacacttacaggatcttctaactggcctggagaaaaaatgaataataattcaaattggtctaacattacttttgatcttccaaatggagtttcaccaaataatattggtgtagtatttaataatggtaacggacaacaaacagttgatttaagcagaggaactgatggatggtttagaattactgatcaaactaatggtaaatatacaggaatatggacccaagattgtcctactaattgtaacagctcaaaaaatgatgctataacacctaaaaacaatactaataaacacttttaccttgcaccaaaccctattatatcaaacgctaacctttatatcagcacagataaaaaaggaactttaaaagtgagtttaactaatattttaggacaaacatcagttattgttaacgaaaaaaaacaatctgggcttcacaagatacctctatctgattctgatttctcaaataaaggtttgttttttattaatgtaacgttagatgatattcaaatagcatcacctttaaaaattattaaacaataa

SEQ ID NO: 2 - protein, 428 aa, Tenacibaculum geojense
MMKQCLKKLLLSLLFINAINISAQDEDVLFQAFDWNVQNQPTGQTWFNVVTQNSSEISNAGFDMIWLPPVSDSGAPQGYLPRELYNFNSAYGTESQLRNLINTYHNLDVKIIGDIVINHRVGTNDAVTFTNPSWPTTFITADDEGRNFVNYPVEFSINGDYFPGTALKADGSNGTYGPARDLDHYNPAVRQEIKNWMNFLKNDLGFDGWRYDFVHGYDPIFNKEYNDATNPYFAVGELLESSRIQTNNWINFTQQSSSAFDFNTKVTLQNALRDNNLSYLRDGNGKPSGMIGINPTKSVTFLDNHDTGAAQQCCGSGYVFPSDENNLRKGYAYILTHPGNPMVFWTHYFDAPIGVKNAIKDLISIRKDARVFAGSTINIAEARNDLYAAYIDGRNGTIAMKLGRGNWSPNGNNWVLRTSGNDYAVWTQ

SEQ ID NO: 3 - DNA, 43 bp, Artificial sequence
  Synthetic construct
tgcctcattctgcagccgcgcaagacgaagatgtactatttca

SEQ ID NO: 4 - DNA, 44 bp, Artificial sequence
  Synthetic construct
tcattagtggtgatggtgatgatgttgtgtccaaacagcataat

SEQ ID NO: 5 - DNA, 2595 bp, Tenacibaculum sp-62066
  CDS (1)..(1293)
  sig_peptide (1)..(66)
  mat_peptide (67)..(1293)
atgaaacaaaccctaaaaaactttcaattagttttgctattcattttttatataaatgtattagcacaagatgaagatgtacttttccaagcttttgattggaatgtacaaaatcaaccagcaggccaaacttggttcgatgttgttacccaaaacagcagtgaaataagtagtgcaggatttgatatgatttggcttccacctcttagtgattctggagctcctcaaggatatcttccaagagagttatacaattttaacagtgcttatggatcagaagttcaactaagaaaccttattaatacgtatcacaacgctggaataaaaattattggtgatattgttatcaatcatagagttggtactaatgatgctgtaacttttacaaatccttcatggccgactacttttattaccgctgatgatgaaggtagaaattttgttaactaccctgttgaatttagtataaacggagactattttcctggaactgcacttaaagcagacggatcaaacggaacgtatggacctgctagagatttagatcataaaaacccagctgttcgtcaagaaatcaaaaactggatgaatttcttgaaaaatgatttaggtcttgatggttggagatatgattttgttcatggttatgatcctatttataacaaagaatataatgatgctacaaacccatattttgcagtgggtgaactattagaaagtagtagagttcaaacaaataactgggtaaactttacacaacaatcttcttctgcttttgattttaacacaaaagttactttacaaaatgctttaagagacaacaacctttcttatttaagagatggtaatggaaatccttcgggcatgattggaattaacgctacaaaatctgtaacttttttagaaaatcatgatacgggagcagcacaacaatgttgtggacctggttatgtgtttcctagtgatgaaactaatttaagaaaaggatatgcatatattttaacacatccagggaatccattggtattctggactcattatttcgatactggaacaggagttagaaatgcaattaaagatttaattgctattcgtaaagatgttagaatatttgcaggatcttctataaacattgatgaagctagaaacgatttatacgctgcatatatagatggtagaaatggaactattgctatgaaattaggaagtggaaactgggctccaaatggaactggatgggttttaagaacatctggaactgattatgctgtttggacgcaaggaggaactcctcctccacctccaacacaagttgatccttttactgtaaactttaaaaaaccaaactcatggagtaataacattaatgtatatctttttgatgcttctacaaatgcaatcattccaggaacatcatcttggccgggacagtcaacgacgaatattgcaggaactccttggtattcttttcaagtaaatcctacaattggagtagcagctcaaaatatcaaaattatttttaatgatggtattaaccaaactgatgatttaattagaagtacaggtggttggtacgataacggaacatggacaaataattgtccttctgattgcacaagtactcctccaccgcctcctttaggaaatgttacttatcaatttaaaaaaccaagtaactggagttctaatgtaaatgcatatttcttcgatgctgctaccaactctacaatttctggaactcaaggatggccaggacaagcaatgtcaagtatttctggaacaccttggtataagtatactttaaatgtaccgtctggaacttcttcaattaatataagagtcatttttaatgatggtgctaaccaaacagatgatttagcgagagaaacagacggatggtatgataatggaacttggacaaatgattgtccatcaaattgtacaaatacgtctcctccatccagtaatatagttactattaactttttaagaacttcttcttgggggaatacagtaaatgcttatttgtataataaaactacgaacacaatgctttctggaacagcaggatggccaggacaacaaatgaataacggaacaaattggtctaattatacgtttacaattccaaatggagtttcatcaaatgatattggtgtagttttcaataacggaaacggacaacaaacagttgatttaacaagaggaactgatggatggtttagaataactggaagtagtaatggaaaatcaacaggagtttggtctgatagttgtcctacaaattgtacttcttcaagaaactcggttaacacaacttctgagttaacaattttggaaaatgtttcaattgcaccaaatccaatacgtaatagatcaaatctttacatttcaacaaataaaaaaggagttttaaaagttagtatatcgaatatactaggtcaaacaaaaataatttttaatggagaaaagagctctggaaaccatcaaatagaattatcaaacgatgactttggagctaagggtatttatatcatcaattcaactttgaataatatcccaatttctaaaccaataaaggttattaaacagtaa

SEQ ID NO: 6 - protein, 431 aa, Tenacibaculum sp-62066
MKQTLKNFQLVLLFIFYINVLAQDEDVLFQAFDWNVQNQPAGQTWFDVVTQNSSEISSAGFDMIWLPPLSDSGAPQGYLPRELYNFNSAYGSEVQLRNLINTYHNAGIKIIGDIVINHRVGTNDAVTFTNPSWPTTFITADDEGRNFVNYPVEFSINGDYFPGTALKADGSNGTYGPARDLDHKNPAVRQEIKNWMNFLKNDLGLDGWRYDFVHGYDPIYNKEYNDATNPYFAVGELLESSRVQTNNWVNFTQQSSSAFDFNTKVTLQNALRDNNLSYLRDGNGNPSGMIGINATKSVTFLENHDTGAAQQCCGPGYVFPSDETNLRKGYAYILTHPGNPLVFWTHYFDTGTGVRNAIKDLIAIRKDVRIFAGSSINIDEARNDLYAAYIDGRNGTIAMKLGSGNWAPNGTGWVLRTSGTDYAVWTQGGTP

SEQ ID NO: 7 - DNA, 1329 bp, artificial sequence
  synthetic construct
atgaagaaaccgttggggaaaattgtcgcaagcaccgcactactcatttctgttgcttttagttcatcgatagcatcggctcaagatgaagatgttctgtttcaagcgtttgattggaatgtccaaaatcaaccggcaggccaaacatggtttgatgttgttacacaaaacagcagcgaaatttcatcagcaggctttgatatgatttggctgcctccgctgtcagattcaggcgcaccgcaaggctatctgccgagagaactgtataactttaattcagcatatggcagcgaagtccaactgcgcaatctgattaatacatatcataatgcaggcattaaaatcattggcgatattgtcattaaccatcgggttggcacaaatgatgcagtcacatttacaaatccgtcatggccgacaacatttattacagcagatgatgaaggcagaaactttgtcaattatccggtcgaatttagcatcaacggcgattattttccgggaacagcactgaaagcagatggctcaaatggcacatatggaccggcaagagatctggatcataaaaatccggcagttcgccaagaaatcaaaaattggatgaattttctgaaaaatgatctgggcctggatggctggcgctatgattttgttcatggctatgatccgatttataacaaagaatacaacgatgcgacgaacccgtattttgcagttggcgaacttctggaatcaagcagagttcaaacaaataactgggtcaactttacacaacaaagctcaagcgcgtttgattttaacacaaaagtcacactgcaaaatgcgctgcgcgataataatctgtcatatctgagagatggcaatggcaatccgtcaggcatgattggcattaatgcaacaaaaagcgtcacatttctggaaaaccatgatacaggcgcagcacaacaatgctgcggaccgggatatgtttttccgtcagatgaaacaaatctgcgcaaaggctatgcgtatattctgacacatccgggaaatccgctggttttttggacacattattttgatacaggcacaggcgttcgcaatgcaattaaagatctgattgcaatccgcaaagatgtcagaatttttgcaggcagcagcattaacattgatgaagcaagaaatgatctgtatgcagcgtatattgatggcagaaatggcacaattgcaatgaaactgggctcaggcaattgggcaccgaatggcacaggctgggttctgagaacatcaggcacagattatgcagtttggacacaaggcggaacaccgcatcatcaccatcaccactaa

SEQ ID NO: 8 - protein, 442 aa, artificial sequence
  Synthetic Construct
MKKPLGKIVASTALLISVAFSSSIASAQDEDVLFQAFDWNVQNQPAGQTWFDVVTQNSSEISSAGFDMIWLPPLSDSGAPQGYLPRELYNFNSAYGSEVQLRNLINTYHNAGIKIIGDIVINHRVGTNDAVTFTNPSWPTTFITADDEGRNFVNYPVEFSINGDYFPGTALKADGSNGTYGPARDLDHKNPAVRQEIKNWMNFLKNDLGLDGWRYDFVHGYDPIYNKEYNDATNPYFAVGELLESSRVQTNNWVNFTQQSSSAFDFNTKVTLQNALRDNNLSYLRDGNGNPSGMIGINATKSVTFLENHDTGAAQQCCGPGYVFPSDETNLRKGYAYILTHPGNPLVFWTHYFDTGTGVRNAIKDLIAIRKDVRIFAGSSINIDEARNDLYAAYIDGRNGTIAMKLGSGNWAPNGTGWVLRTSGTDYAVWTQGGTPHHHHHH

SEQ ID NO: 9 - protein, 415 aa, artificial sequence
  synthetic construct
QDEDVLFQAFDWNVQNQPAGQTWFDVVTQNSSEISSAGFDMIWLPPLSDSGAPQGYLPRELYNFNSAYGSEVQLRNLINTYHNAGIKIIGDIVINHRVGTNDAVTFTNPSWPTTFITADDEGRNFVNYPVEFSINGDYFPGTALKADGSNGTYGPARDLDHKNPAVRQEIKNWMNFLKNDLGLDGWRYDFVHGYDPIYNKEYNDATNPYFAVGELLESSRVQTNNWVNFTQQSSSAFDFNTKVTLQNALRDNNLSYLRDGNGNPSGMIGINATKSVTFLENHDTGAAQQCCGPGYVFPSDETNLRKGYAYILTHPGNPLVFWTHYFDTGTGVRNAIKDLIAIRKDVRIFAGSSINIDEARNDLYAAYIDGRNGTIAMKLGSGNWAPNGTGWVLRTSGTDYAVWTQGGTPHHHHHH

SEQ ID NO: 10 - DNA, 2595 bp, Ahrensia sp-62069
  CDS (1)..(1293)
  sig_peptide (1)..(66)
  mat_peptide (67)..(1293)
atgaaacaaaacctcaaaaactttcaattttttttgctattcattttttccctgaatgttctagcacaagacgaagacgtgctctttcaggcatttgactggaatgtacaaaatcaaccagccggacaaacttggttcgatgttgttactcaaaatagcaatgaaatctccagtgctggctttgatatgatttggctaccaccggtaagtgattctggagctcctcaaggatatttacctagagaattgtataactttaatagtgcgtatggatcagaagtacaattaagaaatttaattaacacctatcacaacgctggagtaaaaattatcggtgatattgttatcaatcatcgtgtaggaaccaacgatgcagttactttcacaaatccagcttggcctactacgttcattacatcagatgatgaaggaagaaattttgtaaactttcctgtagagtttagtattaatggagattattttcccggtaatgctttgaaagctgatggatcgaatggaacttatggaccagcaagagatttagaccataaaaatcctgctgttcgtcaagaaattaaaaactggatgaacttcttaaaaaatgatctaggttttgacggatggagatatgattttgttcatggatacgatcctatttacaataaggaatataatgatgctacaaatccatacttcgcagttggtgaattattagaaagtagtagagttcaaactaataattgggtaaactttacccaacaatcatcttctgcttttgatttcaatactaaagttaccttacagaatgctcttagagataataatctttcttatttgagagatgggagtggtaacccttcaggaatgattggaattaaccctgcaaaatcggtaacttttttagaaaatcatgatacaggagcagcacaacaatgttgtggacctgattatgtatttcctggtgatgaaactaatttaagaaaaggatatgcatatattttatcgcacccaggaaatccaatggtattttggacacattattttaacggaggaactggcgtaagaaatgcaattaaagatatgattgctattcgtaaagatgcaagaatttttgcaggatcttctataaacattgctgaagcaagaaacgatttatatgcagcttatatagatggaagaaatgggacaattgctatgaaattaggaagtggaaactgggctccaaacggaactggatgggttttaagaacatctggaactgattatgcagtttggactcaaggaggaactccaccaccaccaacacaagttgattcttttacagtaaactttaaaaaacctaattcttggaattctaatgtaaatgcatatttatttgatgcttcaacaaacgctataattccagggacaactggatggccaggagaatctatgacaaatattactggaactccttggtattcttttcaagttaatcctacctctggagtggctgcagaaaatattagaataatttttaacgatggcacaaatcaaacggatgatctaagcagaagtattgatggttggtatgataacggaaattggacaaataattgtccttctgattgtactggaactcctccaccgccccctccaccatctggaaacttaactgttcaatataaaaagcctaataactggagctcaaatattaatgtgtacttctttgatgtttcaacaaatagtacaattcctggaacttcaggttggccaggagctacaatgactaacatttctggtactccttggtataaattcactcttaatgtaccttctgagacatcatcaaataatatcagaatgattttcaatgatggaaataaccagactgatgatttagcgagaggttctgatggatggtataacaatggaatttggactagtaattgtccttctgattgcactagcacaccaccaacaaatactaaagcaacgattaacttcttaagaacttcttcttggggaaatactgtaaatgcttacttgtataacaaaactacaaattcaacactttctggaacaccaggatggccaggtcagcaaatgaataatactgcagattggtctaattacacgtttactattcctgatggcgtagcatctaatgatattggcgtagtttttagtaatggaaacggacaacaaacagttgatttaactcgaggaactgatggttggttcagaatcacgggaagtagtaatggaaaatcaacaggtgtatggacagataactgcactacgagttgtacatcttctagaggttcaataaacgttagaaccgaattatccattttaaacaatatttcgattgcccctaatccgataagaaacaaatcaagaatttatatctctaccagtagaaaaggaatattaaaaattagtatttcaaatatactaggtcagaccaaagtcatttacaataatgaaaagcaaaaaggtaatcatcaaatagaattatcagatgctgattttggagctaaaggaatttatattataaattctactttgaatgatactcctatttcaaaaccaataaaagttattaaacagtaa

SEQ ID NO: 11 - protein, 431 aa, Ahrensia sp-62069
MKQNLKNFQFFLLFIFSLNVLAQDEDVLFQAFDWNVQNQPAGQTWFDVVTQNSNEISSAGFDMIWLPPVSDSGAPQGYLPRELYNFNSAYGSEVQLRNLINTYHNAGVKIIGDIVINHRVGTNDAVTFTNPAWPTTFITSDDEGRNFVNFPVEFSINGDYFPGNALKADGSNGTYGPARDLDHKNPAVRQEIKNWMNFLKNDLGFDGWRYDFVHGYDPIYNKEYNDATNPYFAVGELLESSRVQTNNWVNFTQQSSSAFDFNTKVTLQNALRDNNLSYLRDGSGNPSGMIGINPAKSVTFLENHDTGAAQQCCGPDYVFPGDETNLRKGYAYILSHPGNPMVFWTHYFNGGTGVRNAIKDMIAIRKDARIFAGSSINIAEARNDLYAAYIDGRNGTIAMKLGSGNWAPNGTGWVLRTSGTDYAVWTQGGTP

SEQ ID NO: 12 - DNA, 1326 bp, artificial sequence
  synthetic construct
atgaagaaaccgttggggaaaattgtcgcaagcaccgcactactcatttctgttgcttttagttcatcgatagcatcggctcaagatgaagatgttctgtttcaagcgtttgattggaatgtccaaaatcaaccggcaggccaaacatggtttgatgttgttacacagaacagcaacgaaatttcatcagcaggctttgatatgatttggctgcctccggtttcagattcaggcgcaccgcaaggctatctgccgagagaactgtataactttaattcagcatatggcagcgaagtccaactgcgcaatctgattaatacatatcataacgcaggcgtcaaaatcattggcgatattgtcattaatcatcgggtcggcacaaatgatgcagtcacatttacaaatccggcatggccgacaacatttatcacatcagatgatgaaggcagaaactttgtcaattttccggtcgaatttagcatcaacggcgattattttccgggaaatgcactgaaagcagatggctcaaatggcacatatggaccggcaagagatctggatcataaaaatccggcagttcgccaagaaatcaaaaattggatgaattttctgaaaaatgatctgggctttgatggctggcgctatgattttgttcatggctatgatccgatttataacaaagaatacaacgatgcgacgaacccgtattttgcagttggcgaacttctggaatcaagcagagttcaaacaaataactgggtcaactttacacaacaaagctcaagcgcgtttgattttaacacaaaagtcacactgcaaaatgcgctgcgcgataataatctgtcatatctgagagatggctcaggcaatccgtcaggcatgattggcattaatccggcaaaatcagttacatttctggaaaaccatgatacaggcgcagcacaacaatgctgcggaccggattatgtttttccgggtgatgaaacaaatctgagaaaaggctatgcgtatattctgtcacatccgggaaatccgatggttttttggacgcattactttaatggcggaacaggcgttagaaacgcgattaaagatatgattgcgattcgcaaagatgcacgcatttttgcaggctcatcaattaacattgcggaagcaagaaatgatctgtacgcagcatatattgatggcagaaatggcacaattgcaatgaaactgggcagcggcaattgggcaccgaatggcacaggctgggttctgagaacatcaggcacagattatgcagtttggacacaaggcggaacaccgcatcatcaccatcaccac

SEQ ID NO: 13 - protein, 442 aa, artificial sequence
  Synthetic Construct
MKKPLGKIVASTALLISVAFSSSIASAQDEDVLFQAFDWNVQNQPAGQTWFDVVTQNSNEISSAGFDMIWLPPVSDSGAPQGYLPRELYNFNSAYGSEVQLRNLINTYHNAGVKIIGDIVINHRVGTNDAVTFTNPAWPTTFITSDDEGRNFVNFPVEFSINGDYFPGNALKADGSNGTYGPARDLDHKNPAVRQEIKNWMNFLKNDLGFDGWRYDFVHGYDPIYNKEYNDATNPYFAVGELLESSRVQTNNWVNFTQQSSSAFDFNTKVTLQNALRDNNLSYLRDGSGNPSGMIGINPAKSVTFLENHDTGAAQQCCGPDYVFPGDETNLRKGYAYILSHPGNPMVFWTHYFNGGTGVRNAIKDMIAIRKDARIFAGSSINIAEARNDLYAAYIDGRNGTIAMKLGSGNWAPNGTGWVLRTSGTDYAVWTQGGTPHHHHHH

SEQ ID NO: 14 - protein, 415 aa, Artificial sequence
  synthetic construct
QDEDVLFQAFDWNVQNQPAGQTWFDVVTQNSNEISSAGFDMIWLPPVSDSGAPQGYLPRELYNFNSAYGSEVQLRNLINTYHNAGVKIIGDIVINHRVGTNDAVTFTNPAWPTTFITSDDEGRNFVNFPVEFSINGDYFPGNALKADGSNGTYGPARDLDHKNPAVRQEIKNWMNFLKNDLGFDGWRYDFVHGYDPIYNKEYNDATNPYFAVGELLESSRVQTNNWVNFTQQSSSAFDFNTKVTLQNALRDNNLSYLRDGSGNPSGMIGINPAKSVTFLENHDTGAAQQCCGPDYVFPGDETNLRKGYAYILSHPGNPMVFWTHYFNGGTGVRNAIKDMIAIRKDARIFAGSSINIAEARNDLYAAYIDGRNGTIAMKLGSGNWAPNGTGWVLRTSGTDYAVWTQGGTPHHHHHH

SEQ ID NO: 15 - protein, 485 aa, Bacillus sp AA560
HHNGTNGTMMQYFEWYLPNDGNHWNRLRSDASNLKDKGISAVWIPPAWKGASQNDVGYGAYDLYDLGEFNQKGTIRTKYGTRNQLQAAVNALKSNGIQVYGDVVMNHKGGADATEMVRAVEVNPNNRNQEVSGEYTIEAWTKFDFPGRGNTHSNFKWRWYHFDGVDWDQSRKLNNRIYKFRGDGKGWDWEVDTENGNYDYLMYADIDMDHPEVVNELRNWGVWYTNTLGLDGFRIDAVKHIKYSFTRDWINHVRSATGKNMFAVAEFWKNDLGAIENYLNKTNWNHSVFDVPLHYNLYNASKSGGNYDMRQIFNGTVVQRHPMHAVTFVDNHDSQPEEALESFVEEWFKPLAYALTLTREQGYPSVFYGDYYGIPTHGVPAMKSKIDPILEARQKYAYGRQNDYLDHHNIIGWTREGNTAHPNSGLATIMSDGAGGNKWMFVGRNKAGQVWTDITGNRAGTVTINADGWGNFSVNGGSVSIWVNK